(19) United States
(12) Patent Application Publication
(10) Pub. No.: US 2019/0128895 A1
(43) Pub. Date: May 2, 2019

(54) METHODS AND COMPOSITIONS FOR PROGNOSING PRETERM BIRTH

(71) Applicant: LDX Prognostics Limited Co., Jiangsu (CN)

(72) Inventor: Wenkai XIANG, Belmont, CA (US)

(21) Appl. No.: 16/094,866

(22) PCT Filed: Apr. 20, 2016
(86) PCT No.: PCT/CN2016/079742

§ 371 Date: Oct. 18, 2018

(51) Int. Cl. G01N 33/68

(52) U.S. Cl. CPC G01N 33/689; G01N 2800/52; G01N 2800/368

(57) ABSTRACT

A method, composition and kit for determining whether a pregnant woman is at risk of preterm birth. The method entail measuring the protein expression levels of Activin A, Adam12 and sFlt1 and their upregulation indicates that the woman is at risk of preterm risk. The prognosis can be made for women at different stages of pregnancy, including from 16 weeks to 27 weeks. Once a woman is prognosed to be at risk, suitable treatment procedure can be applied to mitigate the risk.

### TECHNICAL FIELD

The present disclosure relates to methods for prognosing preterm birth and compositions used therefor.

### BACKGROUND

Nearly 11% of all pregnancies in the US are result in preterm birth (<37 weeks gestation), contributing greatly to perinatal morbidity and mortality (Goldenberg, R. L. and Rouse, D. J. (1998). Prevention of premature birth. N Engl J Med 339, 313-20). Etiologies of preterm birth are largely unknown, and predictive biomarkers have yet to be adequately developed.

An increase in the abundance of cervicovaginal fetal fibronectin, assayed by an enzyme linked immunosorbent assay (ELISA) containing FDC-6 monoclonal antibody, has been reported to correlate with an increased likelihood of preterm births. Swabs can be taken from the ectocervix or posterior vaginal fornix, and can be used to detect fetal fibronectin. However, the sensitivity of this biomarker as a predictive tool is relatively low in asymptotic pregnant women: birth <34 weeks, ROC area 0.61 (95% CI: 0.59 to 0.63); birth <37 weeks, ROC area 0.65 (95% CI: 0.63 to 0.66) (Honest et al. BMJ. 325:1-10). In clinical use, in addition, factors such as contamination of the sample with maternal blood, sampling within 24 hours after intercourse, and pre-eclampsia may reduce the accuracy of the test and give false positive results. Thus, there is a need to improve upon this method of predicting preterm birth.

### SUMMARY

The present disclosure relates generally to methods, compositions and kits for determining whether a pregnant woman is at risk of preterm birth. The methods entail measuring the protein expression levels of Activin A, and optionally any one of Adam12 and sFlt1 and their upregulation indicates that the woman is at risk of perterm risk. The prognosis can be made for women at different stages of pregnancy, including from 16 weeks to 27 weeks, 28-31 weeks, and 32-36 weeks. Once a woman is prognosed to be at risk, suitable treatment procedure can be applied to mitigate the risk.

The present disclosure, in one embodiment, provides a method for identifying a pregnant woman as at risk of preterm birth, comprising measuring, in a biological sample isolated from the woman, the expression level of Activin A (inhibin beta A or INHBA); and identifying the woman as at risk of preterm birth if the expression level of of Activin A is upregulated, wherein the upregulation is as compared to a control pregnant woman not at risk of preterm birth.

The present disclosure, in another embodiment, provides a method for identifying a pregnant woman as at risk of preterm birth, comprising measuring, in a biological sample isolated from the woman, an expression representation of Activin A (inhibin beta A or INHBA) and an expression representation of Adam12 (ADAM metallopeptidase domain 12); and identifying the woman as at risk of preterm birth if the expression representations of Activin A and Adam12 are upregulated, wherein the upregulation is as compared to a control pregnant woman not at risk of preterm birth.

The present disclosure, in another embodiment, provides a method for identifying a pregnant woman as at risk of preterm birth, comprising measuring, in a biological sample isolated from the woman, an expression representation of Activin A (inhibin beta A or INHBA) and sFlt1 (fms related tyrosine kinase 1); and identifying the woman as at risk of preterm birth if the expression representations of Activin A and sFlt1 are upregulated, wherein the upregulation is as compared to a control pregnant woman not at risk of preterm birth.

The present disclosure, in another embodiment, provides a method for identifying a pregnant woman as at risk of preterm birth, comprising measuring, in a biological sample isolated from the woman, an expression representation of Activin A (inhibin beta A or INHBA), Adam12 (ADAM metallopeptidase domain 12) and sFlt1 (fms related tyrosine kinase 1); and identifying the woman as at risk of preterm birth if the expression representations of Activin A, Adam12 and sFlt1 are upregulated, wherein the upregulation is as compared to a control pregnant woman not at risk of preterm birth.

It is discovered that the expression levels of the genes Activin A, Adam12 and sFlt1 are the most significant indicator of preterm birth, even though additional genes may be measured. Surprisingly, some genes which were believed to be strong indictors of preterm risks did not appear to be useful in the present disclosure. Such examples include FN1, PEG10, and PAPPA2.

Therefore, in one embodiment, the methods of the present disclosure do not include measurement of expression levels of FN1, PEG10, and/or PAPPA2. In one embodiment, the methods do not include measurement of expression levels of FN1, PEG10, PAPPA2, EPAS1, F5, FBN1, HGF, IGF2, AGO2, ATF2, KDM6A, KRAS, MECOM, PDPK1, S100A8, SPTBN1, TRA2B, VEGFA, WNK1, ACSS1, BMP7, CGB, CYP19A1, DLX4, ELOVL2, EZR, HBB, IL6ST, MFSD2A, PEG3, and/or SVEP1.

In one embodiment, the methods do not include measurement of the expression level of FN1. In one embodiment, the methods do not include measurement of the expression level of PEG10. In one embodiment, the methods do not include measurement of the expression level of PAPPA2. In one embodiment, the methods do not include measurement of the expression level of EPAS1.

In one embodiment, the methods do not include measurement of the expression level of F5. In one embodiment, the methods do not include measurement of the expression level of FBN1. In one embodiment, the methods do not include measurement of the expression level of HGF. In one embodiment, the methods do not include measurement of the expression level of IGF2. In one embodiment, the methods do not include measurement of the expression level of AGO2. In one embodiment, the methods do not include measurement of the expression level of ATF2. In one embodiment, the methods do not include measurement of the expression level of KDM6A. In one embodiment, the methods do not include measurement of the expression level of KRAS. In one embodiment, the methods do not include measurement of the expression level of MECOM. In one embodiment, the methods do not include measurement of the expression level of PDPK1. In one embodiment, the methods do not include measurement of the expression level of S100A8. In one embodiment, the methods do not include measurement of the expression level of SPTBN1. In one embodiment, the methods do not include measurement of the expression level of TRA2B. In one embodiment, the methods do not include measurement of the expression level of VEGFA. In one embodiment, the methods do not include measurement of the expression level of WNK1. In one embodiment, the methods do not include measurement of the expression level of ACSS1. In one embodiment, the methods do not include measurement of the expression level of BMP7. In one embodiment, the methods do not include measurement of the expression level of CGB. In one embodiment, the methods do not include measurement of the expression level of CYP19A1. In one embodiment, the methods do not include measurement of the expression level of DLX4. In one embodiment, the methods do not include measurement of the expression level of ELOVL2. In one embodiment, the methods do not include measurement of the expression level of EZR. In one embodiment, the methods do not include measurement of the expression level of HBB. In one embodiment, the methods do not include measurement of the expression level of IL6ST. In one embodiment, the methods do not include measurement of the expression level of MFSD2A. In one embodiment, the methods do not include measurement of the expression level of PEG3. In one embodiment, the methods do not include measurement of the expression level of SVEP1.

In one embodiment, the measurement is performed for no more than 10 genes, or alternatively no more than 9 genes, 8 genes, 7 genes, 6 genes, 5 genes, 4 genes or 3 genes.

The methods of the present disclosure are suitable for women at different stages of pregnancy, which is unexpected given typically such prognosis is only made for women that are pregnant for more than 32 weeks. In one embodiment, the woman is pregnant for 16-27 weeks. In one embodiment, the woman is pregnant for 28-31 weeks. In one embodiment, the woman is pregnant for 16-31 weeks. In one embodiment, the woman is pregnant for less than 32 weeks. In one embodiment, the woman is pregnant for 32-36 weeks.

The methods may be particularly suitable for certain pregnant women, such as those that smoke or consume alcohol, are younger than 17 or older than 35, have preterm birth history and/or are stressed or unhealthy.

Once the preterm risk is determined, the woman can be subject to a procedure that helps ameliorate the preterm birth risk. Examples of such procedures include, without limitation, administration of corticosteroid, magnesium sulfate, an antibiotic, or progestin, and cervical cerclage and combinations thereof.

The biological sample used can be serum, blood, urine, or any sample that includes a cell or tissue of the woman.

In one embodiment, provided is a method of treating a pregnant woman as at risk of preterm birth, comprising: (a) measuring, in a serum sample obtained from the woman, the expression levels of no more than six genes comprising Activin A (inhibin beta A or INHBA), and optionally any or both of Adam12 (ADAM metallopeptidase domain 12) and sFlt1 (fins related tyrosine kinase 1) and excluding FN1, PEG10, and PAPPA2, wherein the woman is pregnant for less than 37 weeks; and (b) administering to the woman a procedure to ameliorate the preterm birth risk when the woman is identified as at risk of preterm birth for having unregulated expression level of Activin A, wherein the upregulation is as compared to a control pregnant woman not at risk of preterm birth.

In one embodiment, provided is a method of treating a pregnant woman as at risk of preterm birth, comprising: (a) measuring, in a serum sample obtained from the woman, the expression levels of no more than six genes comprising Activin A (inhibin beta A or INHBA) and excluding FN1, PEG10, and PAPPA2, wherein the woman is pregnant for less than 37 weeks; and (b) administering to the woman a procedure to ameliorate the preterm birth risk when the woman is identified as at risk of preterm birth for having unregulated expression level of Activin A, wherein the upregulation is as compared to a control pregnant woman not at risk of preterm birth.

In one embodiment, provided is a method of treating a pregnant woman as at risk of preterm birth, comprising: (a) measuring, in a serum sample obtained from the woman, the expression levels of no more than six genes comprising Activin A (inhibin beta A or INHBA) and Adam12 (ADAM metallopeptidase domain 12) and excluding FN1, PEG10, and PAPPA2, wherein the woman is pregnant for less than 37 weeks; and (b) administering to the woman a procedure to ameliorate the preterm birth risk when the woman is identified as at risk of preterm birth for having unregulated expression levels of Activin A and Adam12, wherein the upregulation is as compared to a control pregnant woman not at risk of preterm birth.

In one embodiment, provided is a method of treating a pregnant woman as at risk of preterm birth, comprising: (a) measuring, in a serum sample obtained from the woman, the expression levels of no more than six genes comprising Activin A (inhibin beta A or INHBA) and sFlt1 (fms related tyrosine kinase 1) and excluding FN1, PEG10, and PAPPA2, wherein the woman is pregnant for less than 37 weeks; and (b) administering to the woman a procedure to ameliorate the preterm birth risk when the woman is identified as at risk of preterm birth for having unregulated expression levels of Activin A and sFlt1, wherein the upregulation is as compared to a control pregnant woman not at risk of preterm birth.

In one embodiment, provided is a method of treating a pregnant woman as at risk of preterm birth, comprising: (a) measuring, in a serum sample obtained from the woman, the expression levels of no more than six genes comprising Activin A (inhibin beta A or INHBA), Adam12 (ADAM metallopeptidase domain 12) and sFlt1 (frns related tyrosine kinase 1) and excluding FN1, PEG10, and PAPPA2, wherein the woman is pregnant for less than 37 weeks; and (b) administering to the woman a procedure to ameliorate the preterm birth risk when the woman is identified as at risk of preterm birth for having unregulated expression levels of Activin A, Adam12 and sFlt1, wherein the upregulation is as compared to a control pregnant woman not at risk of preterm birth.

In one embodiment, the procedure is selected from the group consisting of administration of corticosteroid, magnesium sulfate, an antibiotic, or progestin, and cervical cerclage and combinations thereof.

Also provided are kits or packages useful for performing the methods. In one embodiment, the kit or package comprises: (a) antibodies directed to proteins comprising Activin A (inhibin beta A or INHBA); (b) reagents for detecting protein expression with the antibodies; (c) control antibody and/or control sample. In another embodiment, the kit or package further comprising antibodies direct to proteins comprising any one or both of Adam12 (ADAM metallopeptidase domain 12) and sFlt1 (frns related tyrosine kinase 1). In one embodiment, the kit or package comprises (a) antibodies directed to proteins comprising Activin A (inhibin beta A or INHBA), Adam12 (ADAM metallopeptidase domain 12) and sFlt1 (fins related tyrosine kinase 1); (b) reagents for detecting protein expression with the antibodies; (c) control antibody and/or control sample.

In one embodiment, the kit or package includes only antibodies directed to no more than six proteins (or no more than 5, 4 or 3) beside the control antibody. In some embodiments, the kit or package includes only antibodies directed to Activin A, Adam12 and sFlt1, beside the control antibody.

In one embodiment, the kit or package comprises: (a) oligonucleotides directed to nucleic acid transcripts of gene Activin A (inhibin beta A or INHBA); (b) reagents for detecting nucleic acid transcript with the oligonucleotides; (c) control nucleic acids and/or control sample. In another embodiment, the kit or package further comprises oligonucleotides directed to nucleic acid transcripts of genes comprising any one or both of Adam12 (ADAM metallopeptidase domain 12) and sFlt1 (fins related tyrosine kinase 1). In one embodiment, the kit or package comprises (a) oligonucleotides directed to nucleic acid transcript comprising Activin A (inhibin beta A or INHBA), Adam12 (ADAM metallopeptidase domain 12) and sFlt1 (fins related tyrosine kinase 1); (b) reagents for detecting nucleic acid transcript with the oligonucleotides; (c) control nucleic acids and/or control sample.

In one embodiment, the kit or package includes only oligonucleotides directed to no more than six genes (or no more than 5, 4 or 3) beside the control nucleic acids. In some embodiments, the kit or package includes only oligonucleotides directed to Activin A, Adam12 and sFlt1, beside the control nucleic acids.

Also provided is usse of the kit or package as defined herein in the preparation of a diagonisis composition for diagnosing whether a woman is at risk of preterm birth.

### BRIEF DESCRIPTION OF THE DRAWINGS

FIG. 1. Study outline of the multi-omics based discovery and validation of preterm birth biomarkers. Candidate analytes, from the discovery analyses of differential (preterm birth versus normal control) placenta gene expression analysis, mouse placenta genetics loss of function analysis, and human placenta tissue expression specificity analysis, which failed subsequent validation, were greyed out. Red arrow and green arrow represent up-regulated and down-regulated gene expression, respectively. Superscript: at protein level (circulation); Subscript: RNA level (microarray data).

FIG. 2. Venn diagram analyses of the candidates from the multi-omics based discovery analysis: Preterm differential genes: p-value<0.05, fold change>1.2; Placenta genes: Enriched genes, enhanced gene, and highest expressed gene with FPKM>100 in placenta compared with 32 tissues (Mathias Uhlén et al., 2015, Science); Genetic defect human orthologous genes: abnormal embryonic-extraembryonic boundary morphology MP:0003890 (n=30); abnormal extraembryonic tissue morphology MP:0002086 (n=567); abnormal extraembryonic tissue physiology MP:0004264 (n=34).

FIG. 3. Transcription analysis of the candidate genes for preterm birth. Left panel: Placenta gene expression (unit: FPKM); middle panel: gene expression ratio between placenta and other organ tissues; right panel: gene expression ratio of the placenta tissue between preterm birth and normal controls.

FIGS. 4A-B provide a summary of the validation by ELISA of serological proteins of interest as biomarkers (Activin A) that are predictive of preterm birth. The blood serum samples were collected at different gestational age of the pregnancy: GA<28; GA between 28 and 31; GA between 32 and 37. P values were computed using Mann Whiney Test. A. Box plot of the distribution of the serum ELISA concentrations in case/control and different gestation collection time point samples. B. Top panel: distribution of the subjects of their serum analyte concentration as a function of the gestational age of sample collection; middle panel: distribution of the subjects of their serum analyte concentration as a function of the gestational days from sample collection to delivery; bottom panel: distribution of the subjects of their serum analyte concentration as a function of the gestational days of delivery.

FIGS. 5A-B provide a summary of the validation by ELISA of serological proteins of interest as biomarkers (Adam12) that are predictive of preterm birth. The blood serum samples were collected at different gestational age of the pregnancy: GA<28; GA between 28 and 31; GA between 32 and 37. P values were computed using Mann Whiney Test. A. Box plot of the distribution of the serum ELISA concentrations in case/control and different gestation collection time point samples. B. Top panel: distribution of the subjects of their serum analyte concentration as a function of the gestational age of sample collection; middle panel: distribution of the subjects of their serum analyte concentration as a function of the gestational days from sample collection to delivery; bottom panel: distribution of the subjects of their serum analyte concentration as a function of the gestational days of delivery.

FIGS. 6A-B provides a summary of the validation by ELISA of serological proteins of interest as biomarkers (sFlt1) that are predictive of preterm birth. The blood serum samples were collected at different gestational age of the pregnancy: GA<28; GA between 28 and 31; GA between 32 and 37. P values were computed using Mann Whiney Test. A. Box plot of the distribution of the serum ELISA concentrations in case/control and different gestation collection time point samples. B. Top panel: distribution of the subjects of their serum analyte concentration as a function of the gestational age of sample collection; middle panel: distribution of the subjects of their serum analyte concentration as a function of the gestational days from sample collection to delivery; bottom panel: distribution of the subjects of their serum analyte concentration as a function of the gestational days of delivery.

FIG. 7 demonstrates the performance in predicting preterm birth that is achieved by using the biomarker panel comprising activing A, Adam12, and sFlt-1. Top panel: distribution of the subject biomarker panel score as a function of the sample collection gestational age (weeks); Bottom panel: ROC curves of the biomarker panel were analyzed to compute area under the curve (AUC) values.

It will be recognized that some or all of the figures are schematic representations for purpose of illustration.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

### Methods of Prognosing Preterm Birth

“Preterm birth” or “spontaneous preterm birth” refers to preterm birth, also known as premature birth, which is the birth of a baby at less than 37 weeks gestational age. These babies are known as preemies or premmies. Symptoms of preterm labor include uterine contractions which occur more often than every ten minutes or the leaking of fluid from the vagina. Premature infants are at greater risk for cerebral palsy, delays in development, hearing problems, and problems seeing. These risks are greater the earlier a baby is born. The cause of preterm birth is often not known. Risk factors include diabetes, high blood pressure, being pregnant with more than one baby, being either obese or underweight, a number of vaginal infections, tobacco smoking, and psychological stress, among others. It is recommended that labor not be medically induced before 39 weeks unless required for other medical reasons. The same recommendation applies to cesarean section. Preterm labor and delivery continue to plague modern obstetrics. The preterm birth rate still rests at approximately 11% of deliveries, with ensuing neonatal morbidity and death. This is unchanged despite research into strategies such as tocolytics, risk assessment, and regionalization. Neonatal survival has improved by advances in the neonatal intensive care unit and the use of antepartum steroid administration to reduce the incidence of outcomes (such as respiratory distress syndrome and intraventricular hemorrhage). There has recently been a strong push toward the identification of patients at risk for preterm birth before the onset of labor symptoms. The use of different markers, most notably the presence of bacterial vaginosis, assessment of cervicovaginal fetal fibronectin, and cervical length determined by ultrasound scanning, has been studied in the hopes of targeting those women who are at risk for premature delivery, thereby aiding the clinician in decision making to treat specific patients with different modalities (eg, tocolytics, steroids, antibiotics, cerclage). A serum molecular marker would be advantageous because cervical length, fetal fibronectin, and bacterial vaginosis status involve cervical/vaginal evaluation.

In aspects of the disclosure, methods, kits and reagents are provided for prognosing a preterm birth condition. “Prognosis” as used herein generally includes a prediction of a subject's susceptibility to a disease or disorder, i.e. preterm birth; a determination, or diagnosis, as to whether a subject is presently affected by a disease or disorder, i.e. preterm birth; a prediction for a subject affected by a disease or disorder (e.g., determination of the severity of preterm birth, likelihood that a preterm birth condition will develop into early delivery); a prediction of a subject's responsiveness to treatment for the disease or disorder; and the monitoring a subject's condition to provide information as to the effect or efficacy of therapy. The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease. The terms “individual,” “subject,” “host,” and “patient,” are used interchangeably herein and refer to any mammalian subject for whom diagnosis, treatment, or therapy is desired, particularly humans.

In practicing the subject methods, a sample from an individual, e.g., cells or fluid thereof, e.g., blood or serum, is evaluated to obtain an expression representation of one or more preterm birth genes. By an “expression representation” is meant a representation of the expression levels of one or more genes at the RNA or protein level. By a “gene” or “recombinant gene” it is meant a nucleic acid comprising an open reading frame that encodes for a gene product, e.g. an RNA or polypeptide, of interest, e.g. an RNA or polypeptide associated with preterm birth. The boundaries of the coding sequence are determined by a start codon at the 5′ (amino) terminus and a translation stop codon at the 3′ (carboxy) terminus. A transcription termination sequence may be located 3′ to the coding sequence. In addition, a gene may optionally include its natural promoter (i.e., the promoter with which the exons and introns of the gene are operably linked in a non-recombinant cell, i.e., a naturally occurring cell), and associated regulatory sequences, and may or may not have sequences upstream of the AUG start site, and may or may not include untranslated leader sequences, signal sequences, downstream untranslated sequences, transcriptional start and stop sequences, polyadenylation signals, translational start and stop sequences, ribosome binding sites, and the like. By a “preterm birth gene” it is meant a gene that is differentially expressed or protein cofactor that is differentially present in an individual that will develop or has developed preterm birth as compared to an individual that will deliver normally. In other words, the gene product, i.e. mRNA or protein produced from the gene, or protein cofactor, is present at different levels in a sample from individual that will develop or has developed preterm birth as compared to a healthy individual.

As demonstrated in the examples below, the inventors have identified a number of genes and one protein cofactor that find use as preterm birth genes in the subject methods. These include, but are not limited to, Inhibin, Beta A (Activin A, GenBank Accession No. NM_002192); FMS-like tyrosine kinase 1 or sFlt-1; Genbank Accession Nos. NM_001159920.1 (isoform 2), NM_001160030.1 (isoform 3), and NM_001160031.1 (isoform 4)); and ADAM metallopeptidase domain 12 (Adam12, GenBank Accession No. NP_001275903.1, NM_001288974.1 [O43184-4]; NP_001275904.1. NM_001288975.1. [O43184-3], NP_003465.3, NM_003474.5, [O43184-1], NP_067673.2. NM_021641.4. [O43184-2]. Any convenient tissue sample that demonstrates the differential expression in a patient with preterm birth of the one or more preterm birth genes disclosed herein may be evaluated in the subject methods. Typically, a suitable sample source will be derived from fluids into which the product of the preterm birth gene of interest has been released. Sample sources of particular interest include blood samples or preparations thereof, e.g., whole blood, or serum or plasma, and urine. A sample volume of blood, serum, or urine between about 2 μl to about 2,000 μl is sufficient for determining the level of a preterm birth gene product. Generally, the sample volume will range from about 10 μl to about 1,750 μl, from about 20 μl to about 1,500 μl, from about 40 μl to about 1,250 μl, from about 60 μl to about 1,000 μl, from about 100 μl to about 900 μl, from about 200 μl to about 800 μl, from about 400 μl to about 600 μl. In many embodiments, a suitable initial source for the human sample is a blood sample. As such, the sample employed in the subject assays is generally a blood-derived sample. The blood derived sample may be derived from whole blood or a fraction thereof, e.g., serum, plasma, etc., where in some embodiments the sample is derived from blood, allowed to clot, and the serum separated and collected to be used to assay.

In embodiments in which the sample is a serum or serum-derived sample, the sample is generally a fluid sample. Any convenient methodology for producing a fluid serum sample may be employed. In many embodiments, the method employs drawing venous blood by skin puncture (e.g., finger stick, venipuncture) into a clotting or serum separator tube, allowing the blood to clot, and centrifuging the serum away from the clotted blood. The serum is then collected and stored until assayed. Once the patient derived sample is obtained, the sample is assayed to determine the level of preterm birth gene product.

The subject sample may be treated in a variety of ways so as to enhance detection of the preterm birth gene product. For example, where the sample is blood, the red blood cells may be removed from the sample (e.g., by centrifugation) prior to assaying. Such a treatment may serve to reduce the non-specific background levels of detecting the level of a preterm birth gene product using an affinity reagent. Detection of a preterm birth gene product may also be enhanced by concentrating the sample using procedures well known in the art (e.g. acid precipitation, alcohol precipitation, salt precipitation, hydrophobic precipitation, filtration (using a filter which is capable of retaining molecules greater than 30 kD, e.g. Centrim 30™), affinity purification). In some embodiments, the pH of the test and control samples will be adjusted to, and maintained at, a pH which approximates neutrality (i.e. pH 6.5-8.0). Such a pH adjustment will prevent preterm birth gene product complex formation, thereby providing a more accurate quantitation of the level of preterm birth gene product in the sample. In embodiments where the sample is urine, the pH of the sample is adjusted and the sample is concentrated in order to enhance the detection of the preterm birth gene product.

The subject sample is typically obtained from the individual during the second or third trimester of gestation. By “gestation” it is meant the duration of pregnancy in a mammal, i.e. the period of development in the uterus from conception until birth. The time interval of a gestation plus two weeks, i.e. to the last menstrual period, is called the gestation period. Human gestation can be divided into three trimesters, each three months long. The first trimester is from the last menstrual period to the 13th week, the second trimester is from the 14th to 27th week, and the third trimester is from the 28th week to 42 weeks. A subject sample may be obtained early in gestation, for example, on or before 34 weeks of gestation, e.g. at weeks 20-34 of gestation, at 24-34 weeks of gestation, at weeks 30-34 weeks of gestation. The subject sample may be obtained late in gestation, for example, after 34 weeks of gestation, e.g. at week 35, week 36, week 37, week 38, week 39, week 40, week 41, or week 42.

The expression level of one or more preterm birth genes in the subject sample may be evaluated by any convenient method. For example, preterm birth gene expression levels may be detected by measuring nucleic acid transcripts, e.g. mRNAs, of the one or more preterm birth genes, e.g. an RNA expression signature; or by measuring levels of one or more different proteins/polypeptides that are expression products of one or more genes of interest, e.g. a proteomic expression signature. The terms “expression representation” and “gene expression representation” are used broadly to refer to the determination of the expression of one or more genes and/or protein cofactors at the RNA level or protein level. The terms “evaluating”, “assaying”, “measuring”, “assessing,” and “determining” are used interchangeably to refer to any form of measurement, including determining if an element is present or not, and including both quantitative and qualitative determinations. Evaluating may be relative or absolute.

For example, in some embodiments, the expression of genes may be evaluated by obtaining a nucleic acid expression signature. By a “nucleic acid expression representation” is meant a nucleic acid level determination, where the amount or level of one or more nucleic acids, e.g. the nucleic acid transcript of the one or more genes of interest, in the sample is determined. In these embodiments, the sample that is assayed to generate the expression representation is a nucleic acid sample. The nucleic acid sample includes a plurality or population of distinct nucleic acids that includes the expression information of the phenotype determinative genes of interest of the cell or tissue being diagnosed. The nucleic acid may include RNA or DNA nucleic acids, e.g., mRNA, cRNA, cDNA etc., so long as the sample retains the expression information of the host cell or tissue from which it is obtained. The sample may be prepared in a number of different ways, as is known in the art, e.g., by mRNA isolation from a cell, where the isolated mRNA is used as is, amplified, employed to prepare cDNA, cRNA, etc., as is known in the differential expression art. The sample is typically prepared from a cell or tissue harvested from a subject to be diagnosed, e.g., via a blood draw or biopsy of tissue, using standard protocols, where cell types or tissues from which such nucleic acids may be generated include any tissue in which the expression pattern of the to be determined phenotype exists, including, but not limited to, peripheral blood lymphocyte cells, etc., as reviewed above.

The expression representation may be generated from the initial nucleic acid sample using any convenient protocol. While a variety of different manners of detecting nucleic acids are known, such as those employed in the field of differential gene expression analysis, one representative and convenient type of protocol for generating expression representations is array-based gene expression profiling protocols. Such applications are hybridization assays in which a nucleic acid that displays “probe” nucleic acids for each of the genes to be assayed/profiled in the expression representation to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be practiced to generate the expression representations employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., expression representation (e.g., in the form of a transcriptosome), may be both qualitative and quantitative.

Alternatively, non-array based methods for quantitating the level of one or more nucleic acids in a sample may be employed, including those based on amplification protocols, e.g., Polymerase Chain Reaction (PCR)-based assays, including quantitative PCR, reverse-transcription PCR (RT-PCR), real-time PCR, and the like.

As another example, the expression of the at least one preterm birth genes may be evaluated by making a protein level determination, where the amount or level of one or more proteins/polypeptides in the sample is determined, e.g., the protein/polypeptide encoded by the gene of interest. The terms “protein” and “polypeptide” as used in this application are interchangeable. “Polypeptide” refers to a polymer of amino acids (amino acid sequence) and does not refer to a specific length of the molecule. Thus peptides and oligopeptides are included within the definition of polypeptide. This term also refers to or includes post-translationally modified polypeptides, for example, glycosylated polypeptide, acetylated polypeptide, phosphorylated polypeptide and the like. Included within the definition are, for example, polypeptides containing one or more analogs of an amino acid, polypeptides with substituted linkages, as well as other modifications known in the art, both naturally occurring and non-naturally occurring.

Where the expression representation is a determination of gene expression at the protein level, i.e. a protein expression representation, any convenient protocol for evaluating protein levels may be employed wherein the level of one or more proteins in the assayed sample is determined. For example, one representative and convenient type of protocol for assaying protein levels is ELISA. In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hrs at temperatures on the order of about 25°-27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hr at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatograpic column or filter with a wash solution or solvent.

Alternatively, non-ELISA based-methods for measuring the levels of one or more proteins in a sample may be employed. Representative examples include but are not limited to mass spectrometry, proteomic arrays, xMAP™ microsphere technology, flow cytometry, western blotting, and immunohistochemistry.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

The resultant data provides information regarding expression for each of the genes that have been probed, wherein the expression information is in terms of whether or not the gene is expressed and, typically, at what level, and wherein the expression data may be both qualitative and quantitative.

Once the expression level of the one or more preterm birth genes has been determined, the measurement(s) may be analyzed in any of a number of ways to obtain a preterm birth expression representation. In the broadest sense, the expression representation may be qualitative or quantitative. As such, where detection is qualitative, the methods provide a reading or evaluation, e.g., assessment, of whether or not the target analyte, e.g., nucleic acid or expression product, is present in the sample being assayed. In yet other embodiments, the methods provide a quantitative detection of whether the target analyte is present in the sample being assayed, i.e., an evaluation or assessment of the actual amount or relative abundance of the target analyte, e.g., nucleic acid or protein in the sample being assayed. In such embodiments, the quantitative detection may be absolute or, if the method is a method of detecting two or more different analytes, e.g., target nucleic acids or protein, in a sample, relative. As such, the term “quantifying” when used in the context of quantifying a target analyte, e.g., nucleic acid(s) or protein(s), in a sample can refer to absolute or to relative quantification. Absolute quantification may be accomplished by inclusion of known concentration(s) of one or more control analytes and referencing the detected level of the target analyte with the known control analytes (e.g., through generation of a standard curve). Alternatively, relative quantification can be accomplished by comparison of detected levels or amounts between two or more different target analytes to provide a relative quantification of each of the two or more different analytes, e.g., relative to each other.

For example, the preterm birth expression measurements may be analyzed to produce an expression profile. As used herein, an expression profile is the normalized level of expression of one or more preterm birth genes in a patient sample, for example, the normalized level of serological protein concentrations in a patient sample. An expression profile may be generated by any of a number of methods known in the art. For example, the expression level of each gene may be log₂ transformed and normalized relative to the expression of a selected housekeeping gene, e.g. ABL1, GAPDH, or PGK1, or relative to the signal across a whole microarray, etc. A preterm birth expression profile is one example of a preterm birth representation.

As another example, the preterm birth measurements may be analyzed as a biomarker panel. Predictive members of the biomarker panel may be selected by statistical feature selection process. For example, the panel of analytes may be selected by combining genetic algorithm (GA), and all paired (AP) support vector machine (SVM) or random forest (RF) methods for preterm birth classification analysis. Predictive features are automatically determined, e.g. through iterative GA/(SVM or RF), leading to very compact sets of non-redundant preterm birth-relevant analytes with the optimal classification performance. It is possible these different classifier sets harbor only modest overlapping gene features but have similar levels of accuracy.

As another example, the preterm birth expression measurements may be analyzed to generate a preterm birth signature. A preterm birth signature is a single metric value that represents the weighted expression levels (e.g. serological protein concentrations) of a panel of preterm birth genes, e.g. a set of genes comprising the preterm birth genes disclosed herein or a subset thereof, assayed in a patient sample, where the weighted expression levels are defined by the dataset from which the patient sample was obtained. A preterm birth signature for a patient sample may be calculated by any of a number of methods known in the art for calculating gene signatures. For example, the expression levels of each of the one or more preterm birth genes in a patient sample may be log₂ transformed and normalized, e.g. as described above for generating a preterm birth expression profile. The normalized expression levels for each gene is then weighted by multiplying the normalized level to a weighting factor, or “weight”, to arrive at weighted expression levels for each of the one or more genes. The weighted expression levels are then totaled and in some cases averaged to arrive at a single weighted expression level for the one or more preterm birth genes analyzed. The weighting factor, or weight, may be determined by any statistical machine learning methodology, for example, Principle Component Analysis (PCA), linear regression, support vector machines (SVMs), and/or random forests of the dataset from which the sample was obtained may be used. For example, the analyte level of each preterm birth gene may be log₂ transformed and weighted either as 1 (for those genes that are up-regulated in preterm birth) or −1 (for those genes that are down-regulated in preterm birth), and the ratio between the sum of up-regulated genes as compared to down-regulated analytes determined to arrive at a preterm birth signature. A preterm birth expression signature is another example of a preterm birth expression representation.

As another example, the preterm birth expression measurements may be analyzed to produce a preterm birth score. Like a preterm birth signature, a preterm birth score is a single metric value that represents the sum of the weighted expression levels of one or more preterm birth genes in a patient sample. A preterm birth score may be determined by methods very similar to those described above for a preterm birth signature, e.g. the expression levels of each of the one or more preterm birth genes in a patient sample may be log₂ transformed and normalized, e.g. as described above for generating a preterm birth expression profile; the normalized expression levels for each gene is then weighted by multiplying the normalized level to a weighting factor, or “weight”, to arrive at weighted expression levels for each of the one or more genes; and the weighted expression levels are then totaled and in some cases averaged to arrive at a single weighted expression level for the one or more preterm birth genes analyzed. However, in contrast to a preterm birth signature, the weighted expression levels are defined by a reference dataset, or “training dataset”. Thus, the preterm birth score is defined by a reference dataset.

These methods of analysis may be readily performed by one of ordinary skill in the art by employing a computer-based system, e.g. using any hardware, software and data storage medium as is known in the art, and employing any algorithms convenient for such analysis. For example, data mining algorithmscan be applied through “cloud computing”, smartphone based or client-server based platforms, and the like.

In certain embodiments the expression of only one gene is evaluated to produce an expression representation. In yet other embodiments, the expression of two or more, e.g., about 3 or more, about 5 or more, about 10 or more, or about 15 genes is evaluated. Accordingly, in the subject methods, the expression of at least one gene in a sample is evaluated. In certain embodiments, the evaluation that is made may be viewed as an evaluation of the transcriptosome, as that term is employed in the art.

The expression representation arrived at in this manner finds many uses in prognosing preterm birth. For example, the expression representation may be employed to predict if a pregnant woman will develop preterm birth, to diagnose preterm birth in a pregnant woman, to characterize a diagnosed preterm birth, or to monitor the responsiveness of the pregnant to treatment for preterm birth. In some instances, the measurement of particular combinations of preterm birth markers disclosed herein provides for a preterm birth prognosis that has an improved accuracy over a preterm birth prognosis made using standard methods known in the art, e.g. IFN (Fetal Fibronectin) Test developed by Hologic.

In some embodiments, the expression representation is employed by comparing it to a phenotype determination element, i.e. a preterm birth phenotype determination element, to identify similarities or differences with the phenotype determination element, where the similarities or differences that are identified are then employed to predict if a pregnant woman will develop preterm birth, to diagnose preterm birth in a pregnant woman, to characterize a diagnosed preterm birth, to monitor the responsiveness of the pregnant to treatment for preterm birth, etc. For example, a preterm birth phenotype determination element may be a sample from an individual that has or does not have preterm birth, which may be used, for example, as a reference/control in the experimental determination of the expression representation for a given subject. As another example, a preterm birth phenotype determination element may be an expression representation, e.g. expression profile, expression signature or expression score, that is representative of a preterm birth state and may be used as a reference/control to interpret the expression representation of a given subject. The phenotype determination element may be a positive reference/control, e.g., a sample or expression representation thereof from a pregnant woman that has preterm birth, or that will develop preterm birth, or that has preterm birth that is manageable by known treatments, or that has preterm birth that has been determined to be responsive only to the delivery of the baby. Alternatively, the phenotype determination element may be a negative reference/control, e.g. a sample or expression representation thereof from a pregnant woman that has not developed preterm birth, or a woman that is not pregnant. Phenotype determination elements are preferably the same type of sample or, if expression representations, are obtained from the same type of sample as the sample that was employed to generate the expression representation for the individual being monitored. For example, if the serum of an individual is being evaluated, the reference/control would preferably be of serum.

In certain embodiments, the obtained expression representation is compared to a single phenotype determination element to obtain information regarding the individual being tested for preterm birth. In certain embodiments, the obtained expression representation is compared to two or more phenotype determination elements. For example, the obtained expression representation may be compared to a negative reference and a positive reference to obtain confirmed information regarding if the individual will develop preterm birth. As another example, the obtained expression representation may be compared to a reference that is representative of a preterm birth that is responsive to treatment and a reference that is representative of a preterm birth that is not responsive to treatment to obtain information as to whether or not the patient will be responsive to treatment.

The comparison of the obtained expression representation and the one or more phenotype determination elements may be performed using any convenient methodology, where a variety of methodologies are known to those of skill in the art. For example, those of skill in the art of arrays will know that array profiles may be compared by, e.g., comparing digital images of the expression profiles, by comparing databases of expression data, etc. Patents describing ways of comparing expression profiles include, but are not limited to, U.S. Pat. Nos. 6,308,170 and 6,228,575, the disclosures of which are herein incorporated by reference. Methods of comparing expression profiles are also described above. Similarly, those of skill in the art of ELISAs will know that ELISA data may be compared by, e.g. normalizing to standard curves, comparing normalized values, etc. The comparison step results in information regarding how similar or dissimilar the obtained expression profile is to the control/reference profile(s), which similarity/dissimilarity information is employed to prognose preterm birth, for example to predict the onset of a preterm birth, diagnose preterm birth, monitor a preterm birth patient, etc. Similarity may be based on relative expression levels, absolute expression levels or a combination of both. In certain embodiments, a similarity determination is made using a computer having a program stored thereon that is designed to receive input for a gene level expression result obtained from a subject, e.g., from a user, determine similarity to one or more reference profile, and return an preterm birth prognosis, e.g., to a user (e.g., lab technician, physician, pregnant individual, etc.). Further descriptions of computer-implemented aspects of the disclosure are described below.

Depending on the type and nature of the reference/control profile(s) to which the obtained expression profile is compared, the above comparison step yields a variety of different types of information regarding the cell/bodily fluid that is assayed. As such, the above comparison step can yield a positive/negative prediction of the onset of preterm birth. Alternatively, such a comparison step can yield a positive/negative diagnosis of preterm birth. Alternatively, such a comparison step can provide a characterization of a preterm birth.

In other embodiments, the expression representation is employed directly, i.e. without comparison to a phenotype determination element, to make a prediction, diagnosis, or characterization. For example, a patient may be predicted to develop preterm birth if the concentration of Adam 12 in the patient's serum is about 25.86 ng/ml or greater at less than 28 gestational weeks, 19.37 ng/ml or greater between 28 and 32 weeks, 40.03 mg/ml between 32 and 37 weeks. For other examples, see Table 10.

In some embodiments, other analysis may be employed in conjunction with the aforementioned expression level representation to provide a preterm birth prognosis for the individual. Such analyses are well known in the art, and include, for example, an analysis of blood pressure, weight gain, water retention, and platelet count.

In some embodiments, prognosing preterm birth includes providing a prediction, diagnosis, or characterization of preterm birth. In such embodiments, the prediction, diagnosis, or characterization may be provided by providing, i.e. generating, a written report that includes the artisan's monitoring assessment, i.e. the artisan's prediction of the onset of preterm birth (a “preterm birth prediction”), the artisan's diagnosis of the subject's r preterm birth (a “preterm birth diagnosis”), or the artisan's characterization of the subject's preterm birth (a “preterm birth characterization”). Thus, a subject method may further include a step of generating or outputting a report providing the results of a monitoring assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to a subject monitoring assessment and its results. A subject report includes at least a preterm birth prediction, preterm birth diagnosis, or preterm birth characterization, i.e. a prediction as to the likelihood of a patient developing preterm birth, a diagnosis of preterm birth, or a characterization of the preterm birth, respectively. A subject report can be completely or partially electronically generated. A subject report can further include one or more of: 1) information regarding the testing facility; 2) service provider information; 3) patient data; 4) sample data; 5) an assessment report, which can include various information including: a) reference values employed, and b) test data, where test data can include, e.g., a protein level determination; 6) other features.

The report may include information about the testing facility, which information is relevant to the hospital, clinic, or laboratory in which sample gathering and/or data generation was conducted. Sample gathering can include obtaining a fluid sample, e.g. blood, saliva, urine etc.; a tissue sample, e.g. a tissue biopsy, etc. from a subject. Data generation can include measuring the level of polypeptide concentration for one or more genes that are differentially expressed or present at different levels in preterm birth patients versus healthy individuals, i.e. individuals that do not have and/or do not develop preterm birth. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a patient data section, including patient medical history (which can include, e.g., age, race, serotype, prior preterm birth episodes, and any other characteristics of the pregnancy), as well as administrative patient data such as information to identify the patient (e.g., name, patient date of birth (DOB), gender, mailing and/or residence address, medical record number (MRN), room and/or bed number in a healthcare facility), insurance information, and the like), the name of the patient's physician or other health professional who ordered the monitoring assessment and, if different from the ordering physician, the name of a staff physician who is responsible for the patient's care (e.g., primary care physician).

The report may include a sample data section, which may provide information about the biological sample analyzed in the monitoring assessment, such as the source of biological sample obtained from the patient (e.g. blood, saliva, or type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information generated after processing of the data as described herein. The interpretive report can include a prediction of the likelihood that the subject will develop preterm birth. The interpretive report can include a diagnosis of preterm birth. The interpretive report can include a characterization of preterm birth. The interpretive report can include, for example, the results of a protein level determination assay (e.g., “1.5 nmol/liter ADAM12 in serum”); and interpretation, i.e. prediction, diagnosis, or characterization. The assessment portion of the report can optionally also include a recommendation(s). For example, where the results indicate that preterm birth is likely, the recommendation can include a recommendation that diet be altered, blood pressure medicines administered, etc., as recommended in the art.

It will be readily appreciated that the report can include all or some of the elements above, with the proviso that the report generally includes at least the elements sufficient to provide the analysis requested by the user (e.g. prediction, diagnosis or characterization of preterm birth).

As discussed above, the subject methods find use in providing a preterm birth prognosis for individual, where by “providing a preterm birth prognosis” it is meant predicting if the individual will develop preterm birth, diagnosing the presence or absence preterm birth, and/or characterizing preterm birth. By “predicting if the individual will develop preterm birth”, it is meant determining the likelihood that an individual will develop preterm birth in the next week, in the next 3 weeks, in the next 5 weeks, in the next 2 months, in the next 3 months, e.g. during the remainder of the pregnancy. By “diagnosing preterm birth,” it is meant determining that the individual has developed preterm birth, i.e. a hypertension due to the pregnancy, or pregnancy-induced hypertension. By “characterizing a preterm birth” it is meant determining the extent of preterm birth in the individual, e.g. to monitor the individual, determine therapeutic regimen, etc. as is well known in the art.

The subject methods may be employed with a variety of different types of subjects. In many embodiments, the subjects are within the class mammalian, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the animals or hosts, i.e., subjects (also referred to herein as patients) are humans.

### Reagents, Systems and Kits

Also provided are reagents, systems and kits thereof for practicing one or more of the above-described methods. The subject reagents, systems and kits thereof may vary greatly. Reagents of interest include reagents specifically designed for use in producing the above-described expression representations of preterm birth genes from a sample, for example, one or more gene expression determination elements, e.g. antibodies or peptides for the detection of protein, oligonucleotides for the detection of nucleic acids, etc. In some instances, the gene expression determination element comprises a reagent to detect the expression of a single preterm birth gene, e.g. an antibody, a set of PCR primers, etc. In other instances, the gene expression determination element comprises multiple reagents, each specific for a different preterm birth gene product, e.g. a nucleic acid or protein array, an ELISA plate, a multiplex PCR cocktail, etc., which may be used to detect the expression of more than one preterm birth gene simultaneously, For example, the nucleic acid- or antibody-based detection of the sample nucleic acid or protein, respectively, is coupled with an electrochemical biosensor platform that will allow multiplex determination of these biomarkers for personalized preterm birth care. The term “system” refers to a collection of reagents, however compiled, e.g., by purchasing the collection of reagents from the same or different sources. The term kit refers to a collection of reagents provided, e.g., sold, together.

One type of such reagent is an array of probe nucleic acids in which the phenotype determinative genes of interest are represented. A variety of different array formats are known in the art, with a wide variety of different probe structures, substrate compositions and attachment technologies (e.g., dot blot arrays, microarrays, etc.). Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280.

Another type of reagent that is specifically tailored for generating expression representations of phenotype determinative genes, e.g. preterm birth genes, is a collection of gene specific primers that is designed to selectively amplify such genes (e.g., using a PCR-based technique, e.g., real-time RT-PCR). Gene specific primers and methods for using the same are described in U.S. Pat. No. 5,994,076, the disclosure of which is herein incorporated by reference.

Yet another type of reagent that is specifically tailored for generating expression profiles of phenotype determinative genes e.g. preterm birth genes, is a collection of antibodies that bind specifically to the proteins encoded by such genes, e.g. in an ELISA format, in an xMAP™ microsphere format, on a proteomic array, in suspension for analysis by flow cytometry, by western blotting, and immunohistochemistry. Representative antibodies include DVR1008 (R&D Systems), DAD120 (R&D Systems), and DAC00B (R&D Systems). Methods for using the same are well understood in the art. These antibodies can be provided in solution. Alternatively, they may be provided pre-bound to a solid matrix, for example, the wells of a multi-well dish or the surfaces of xMAP microspheres.

Of particular interest are arrays of probes, collections of primers, or collections of antibodies that include probes, primers or antibodies (also called reagents) that are specific for at least 1 gene/protein selected from the group consisting of VEFGR-1, Adam12, Inhibin, beta, in some instances for a plurality of these genes, e.g., at least 2, 3, 4, 8 or more. The subject probe, primer, or antibody collections or reagents may include reagents that are specific only for the genes/proteins/cofactors that are listed above, or they may include reagents specific for additional genes/proteins/cofactors that are not listed above, such as probes, primers, or antibodies specific for genes/proteins/cofactors whose expression pattern are known in the art to be associated with preterm birth.

The systems and kits of the subject disclosure may include the above-described arrays, gene-specific primer collections, or protein-specific antibody collections. The systems and kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. labeled secondary antibodies, streptavidin-alkaline phosphatase conjugate, chemifluorescent or chemiluminescent substrate, and the like.

The subject systems and kits may also include a phenotype determination element, which element is, in many embodiments, a reference or control sample or expression representation that can be employed, e.g., by a suitable experimental or computing means, to make a preterm birth prognosis based on an “input” expression profile, e.g., that has been determined with the above described gene expression determination element. Representative phenotype determination elements include samples from an individual known to have or not have preterm birth, databases of expression representations, e.g., reference or control profiles, and the like, as described above.

The term “system” refers to a collection of reagents, however compiled, e.g., by purchasing the collection of reagents from the same or different sources. The term kit refers to a collection of reagents provided, e.g., sold, together. In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Identification of Serologic Markers for Preterm Birth Risk

Serologic biomarkers were identified to create a diagnostic tool that can be used for earlier and more specific diagnosis of preterm birth.

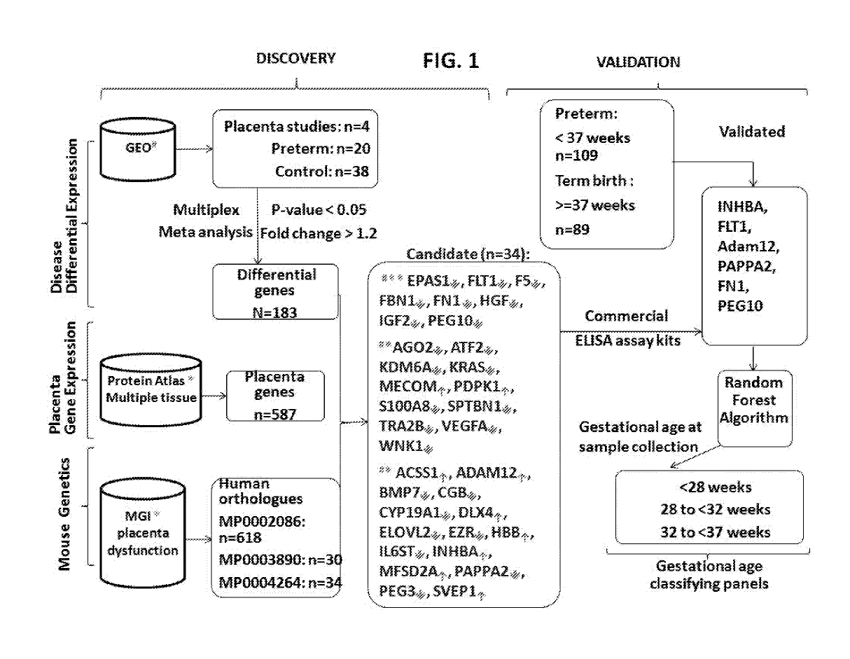

Study design. The overall sample allocation, preterm birth biomarker discovery, validation, and predictive panel construction steps are illustrated in FIG. 1. Our study was conducted in two phases: (1) the discovery phase, which included meta analysis of microarray datasets (three datasets, n=20 preterm birth and n=38 control placenta samples), extraction of placenta specific protein from database of protein atlas and obtainment of human orthologous gene with placenta dysfunction in mouse model from MGI database. (2) the validation phase, which was comprised of the analysis of independent preterm birth (n=109) and control (n=89) cohorts. The candidates for further validation were selected by the fold change >1.2 and p value <0.05 in the meta analysis and by ELISA assay kits available.

Clinical cohort design and sample collection. All the serum samples were procured, with patient consent and institution IRB approvals, from the three teaching hospitals in China. Patients who were diagnosed with suspected placenta previa, cervical cerclage, and trauma precipitating the patient's preterm laboring symptoms (regular uterine contractions, low abdominal cramping, low back pain, pelvic pressure, vaginal bleeding, and increased vaginal discharge) were excluded. Case (preterm birth) and control (normal pregnant) cohorts were matched for gestational age, ethnicity, and parity (details in Tables 1-11).

Multiplex meta-analysis of expression comparing PE and control placentas. As shown in Table 12 below, three PE placenta expression studies (PMID: 22496790; 23290504; 18818296/17170095) were combined and subjected to multiplex meta¬-analysis with the method we previously developed (Morgan et al. Comparison of multiplex meta-analysis techniques for understanding the acute rejection of solid organ transplants. BMC bioinformatics 2010; 11 Suppl 9:S6; Chen et al. Differentially expressed RNA from public microarray data identifies serum protein biomarkers for cross-organ transplant rejection and other conditions. PLoS computational biology 2010; 6). For each of the 22,394 genes tested, we calculated the meta-fold change across all studies. Significant genes were selected if they were measured in 5 or more studies and the meta effect p value was less than 0.05 and the meta-fold change higher than 1.2.

Protein Atlas analysis. According to Uhlen M et al (Proteomics. Tissue-based map of the human proteome. Science. 2015 Jan. 23; 347(6220):1260419.), the placenta genes were extracted from five tissue categories: tissue enriched, group enriched, tissue enhanced, expressed in all (FPKM>100), mixed. (FPKM>100).

Human orthologous gene with placenta dysfunction in mouse model from MGI database. To understand the functional significance of placenta genes in pregnancy disorders, the human orthologous genes whose mouse orthologs were associated with abnormal placental phenotypes when disrupted were obtained from MGI database. Three MGI phenotypes were included: abnormal extraembryonic boundary morphology MP:0003836, abnormal extraembryonic tissue physiology MP:0004264 and abnormal extraembryoic tissue morphology MP:0002086.

ELISA assays validating preterm birth marker candidates. All assays were ELISA assays, and performed using commercial kits following vendors' instructions. All assays were performed to measure serum levels of selected analytes as summarized in Table 13 for the analytes of (VEGFR-1, Adam12, inhibin, beta A, placenta growth factor, fibronectin, paternally expressed 10).

Statistical analyses. Patient demographic and clinical data were analyzed using the “Epidemiological calculator” (R epicalc package). Student's t-test and Mann-Whitney U-test were performed to calculate p values for continuous variables, and Fisher's exact test and Chi-squared test were used for comparative analysis of categorical variables. A group of clinical risk factors of preterm birth were determined by literature review, and their impacts on preterm birth diagnosis were explored by uni- and multivariate analysis. Forest plotting with R rmeta package was used both to represent the placental expression meta analysis and to graphically summarize the serum protein ELISA results. Case (preterm birth) and control samples are not paired; thus the initial serum protein forest plot analysis should be interpreted with caution. Bootstrapping method was used to create “paired” samples from case and control groups for the subsequent forest plotting analysis of the ELISA results. Therefore, serum protein forest plot analysis provides an overall effect estimation of each analyte's capability in discriminating PE and normal pregnant control subjects. Hypothesis testing was performed using Student's t-test (two tailed) and Mann-Whitney U-test (two tailed), and local FDR (Efron et al. Empirical bayes analysis of microarray experiment. J Am Stat Assoc 2001; 96:1151-60) to correct for multiple hypothesis testing issues. Biomarker feature selection and panel optimization was performed using a genetic algorithm (R genalg package). The predictive performance of each biomarker panel analysis was evaluated by ROC curve analysis (Zweig et al. Receiver-operating characteristic (ROC) plots: a fundamental evaluation tool in clinical medicine. Clinical chemistry 1993; 39:561-77; Sing et al. ROCR: visualizing classifier performance in R. Bioinformatics 2005; 21:3940-1). The biomarker panel score was defined as the natural logarithm of the ratio between the geometric means of the respective up- and down-regulated protein biomarkers in the maternal circulation. A composite panel combining all significant biomarkers was developed using random forest algorithm, and evaluated by ROC AUC performance.

Conclusions: Meta-analysis approaches allow for robust identification of top candidate biomarkers that are consistently differentially expressed between preterm birth and normal pregnancy across several studies. Further study of these proteins will yield greater insight into the pathophysiology of preterm birth, and screening methods, reagents, and kits to detect the level of these proteins in patient samples may serve as a point of care diagnostic tool for preterm birth.

TABLE 1
Enrolled pregnant subjects with blood collected at <28 weeks, 28 to <32 weeks, and 32 to <37 weeks.

| | <28 weeks of gestational age | | | | | 28 to <32 weeks of gestational age | | | | |
|---|---|---|---|---|---|---|---|---|---|---|
| | PTB | | Control | | | PTB | | Control | | |
| | N = 4 | | N = 16 | | | N = 56 | | N = 38 | | |
| Characteristic | (20%) | | (80%) | | p value | (59.6%) | | (40.4%) | | p value |
| Ethnicity, n (%) | | | | | NA | | | | | 0.99^{a} |
| Han | 4 | (100) | 16 | (100) | | 55 | (98.2) | 36 | (100) | |
| Zhuang | 0 | | 0 | | | 1 | (1.8) | 0 | | |
| Age (year) | | | | | | | | | | |
| mean (SD) | 25 | (2.9) | 31.1 | (4.6) | 0.022^{b} | 28.9 | (4.4) | 28.4 | (4.3) | 0.62^{b} |
| Education (≤12 | 3 | (75) | 2 | (12.5) | 0.032^{c} | 25 | (44.6) | 12 | (33.3) | 0.39^{c} |
| year), n (%) | | | | | | | | | | |
| Week of gestation at | | | | | | | | | | |
| blood collection | | | | | | | | | | |
| mean (SD) | 25.3 | (1.5) | 24.1 | (3.2) | 0.60^{b} | 29.7 | (1.1) | 29.5 | (1.3) | 0.50^{b} |
| Week of gestation | | | | | | | | | | |
| at delivery | | | | | | | | | | |
| mean (SD) | 28 | (2.6) | 39.5 | (1.2) | <0.001^{b} | 30.9 | (2.3) | 39.3 | (0.9) | <0.001^{b} |
| Days from blood | | | | | | | | | | |
| collection to | | | | | | | | | | |
| delivery | | | | | | | | | | |
| mean (SD) | 15 | (19.3) | 107.8 | (23.2) | <0.001^{b} | 7.7 | (11.5) | 70.3 | (12.6) | <0.001^{b} |

| | 32 to <37 weeks of gestational age | | | | | |
|---|---|---|---|---|---|---|
| | PTB | | Control | | | |
| | N = 42 | | N = 35 | | | Overall |
| Characteristic | (54.5%) | | (45.5%) | | p value | p value |
| Ethnicity, n (%) | | | | | NA | 0.99^{a} |
| Han | 42 | (100) | 35 | (100) | | |
| Zhuang | | | | | | |
| Age (year) | | | | | | |
| mean (SD) | 28.2 | (5.5) | 28 | (4.5) | 0.89^{b} | 0.66^{b} |
| Education (≤12 | 26 | (61.9) | 13 | (40.6) | 0.11^{c} | 0.007^{c} |
| year), n (%) | | | | | | |
| Week of gestation at | | | | | | |
| blood collection | | | | | | |
| mean (SD) | 33.3 | (1.1) | 33.2 | (1.1) | 0.14^{b} | 0.87^{b} |
| Week of gestation | | | | | | |
| at delivery | | | | | | |
| mean (SD) | 33.9 | (1.2) | 39.0 | (1.4) | <0.001^{b} | <0.001^{b} |
| Days from blood | | | | | | |
| collection to | | | | | | |
| delivery | | | | | | |
| mean (SD) | 4.1 | (6.3) | 39.4 | (10.4) | <0.001^{b} | <0.001^{b} |

^{a}Fisher’s exact test;
^{b}Ranksum test;
^{c}Chi-squared test.

TABLE 2
Concurrent medical conditions and clinical features
of the enrolled case and control subjects.

| | PTB | | Control | | |
|---|---|---|---|---|---|
| | N = 102 | | N = 89 | | |
| Characteristic | (53.4%) | | (46.6%) | | p value |
| Concurrent medical conditions/ | | | | | 0.16^{a} |
| Clinical features | | | | | |
| Hyperthyroidism | 1 | (1.0%) | 0 | (0%) | |
| Diabetes (gestational) | 9 | (8.8%) | 3 | (3.4%) | |
| Syphilis | 1 | (1.0%) | 0 | (0%) | |
| Hypothyroidism | 1 | (1.0%) | 3 | (3.4%) | |
| Preterm birth | 1 | (1.0%) | 0 | (0%) | |
| Intrahepatic cholestasis | 1 | (1.0%) | 2 | (2.2%) | |
| Hashimoto's Thyroiditis | 0 | (0%) | 1 | (1.1%) | |
| Gestational hypothyroidism | 0 | (0%) | 2 | (2.2%) | |
| Anemia | 0 | (0%) | 1 | (1.1%) | |
| Pregnancy induced hypertension | 0 | (0%) | 1 | (1.1%) | |
| None | 88 | (86.3%) | 76 | (85.4%) | |

^{a}Fisher's exact test.

TABLE 3
Clinical information of the enrolled case and control subjects.

| Characteristic | Normal (N = 89) | | PTB (N = 102) | | P value |
|---|---|---|---|---|---|
| Concurrent drug, n (%) | | | | | <0.001^{a} |
| Yes | 18 | (20.2) | 81 | (79.4) | |
| Abortion history, n (%) | 30 | (34.5) | 50 | (49) | 0.062^{a} |
| BMI, kg/m², median (IQR) | 20.7 | (19.1, 22.7) | 20.6 | (19.2, 22.7) | 0.84^{b} |
| Nulliparity, n (%) | 49 | (56.3) | 77 | (75.5) | 0.009^{a} |
| Maternal height, cm, mean (SD) | 160.6 | (5.1) | 159.6 | (4.7) | 0.14^{b} |
| Maternal weight, kg, mean (SD) | 54.3 | (7.1) | 53.5 | (7.5) | 0.63^{b} |
| Concurrent diabetes during pregnancy, | 0 | | 6 | (5.9) | 0.031^{c} |
| n (%) | | | | | |
| Proteinuria, n (%) | 1 | (2) | 5 | (6.8) | 0.40^{c} |
| Systolic blood pressure, mmHg, median | 115 | (110, 122.5) | 117 | (110, 125) | 0.45^{b} |
| (IQR) | | | | | |
| Diastolic blood pressure, mmHg, | 70 | (65.5, 77) | 72 | (70, 79) | 0.05^{b} |
| median (IQR) | | | | | |
| WBC, ×10⁹, median (IQR) | 9.9 | (9.1, 12.0) | 11.2 | (9.7, 13.4) | 0.009^{b} |
| Neutrophil, %, median (IQR) | 76.4 | (72.8, 79.2) | 80.3 | (74.3, 84.4) | <0.001^{d} |
| Multiple pregnancy, n (%) | 0 | | 10 | (9.8) | 0.002^{c} |
| Preterm history, n (%) | 0 | | 4 | (3.9) | 0.13^{c} |
| Delivery mode, n (%) | | | | | 0.15^{c} |
| Eutocia | 35 | (40.7) | 44 | (43.6) | |
| Caesarean | 39 | (45.3) | 38 | (37.6) | |
| No | 8 | (9.3) | 18 | (17.8) | |
| Others | 4 | (4.7) | 1 | (1) | |
| Fetus weight, g, mean (SD) | 3350 | (3050, 3650) | 1875 | (1491.2, 2192.5) | <0.001^{d} |
| Fetus height, cm, mean (SD) | 50.1 | (1.6) | 42.7 | (4.6) | <0.001^{d} |
| Total number of pregnancies, n (%) | | | | | 0.097^{a} |
| number = 1 | 49 | (56.3) | 44 | (43.1) | |
| number > 1 | 38 | (43.7) | 58 | (56.9) | |

^{a}Fisher's exact test;
^{b}Ranksum test;
^{c}Chi-squared test.

TABLE 4
Univariate odds ratio analysis of the patient characteristics, without
adjustment of gestational age at blood sample collection.

| | | | | |
|---|---|---|---|---|
| Characteristics | OR | 95CI (L) | 95CI (H) | P value |

4A. Enrolled subjects blood collected <28
weeks of gestational age.

| | | | | |
|---|---|---|---|---|
| WBC, ×10⁹ | 1.21 | 0.95 | 1.65 | 0.1444 |
| BMI prior to pregnancy | 0.83 | 0.45 | 1.3 | 0.4569 |
| (kg/m²) | | | | |
| Education (>12 year) | 0.05 | 0 | 0.56 | 0.0274 |
| Neutrophil (%) | 1.23 | 0.94 | 1.77 | 0.1718 |

4B. Enrolled subjects blood collected between 28
and <32 weeks of gestational age.

| | | | | |
|---|---|---|---|---|
| WBC, ×10⁹ | 1.26 | 1.07 | 1.53 | 0.0114 |
| BMI prior to pregnancy | 1.03 | 0.88 | 1.21 | 0.738 |
| (kg/m²) | | | | |
| Education (>12 year) | 0.62 | 0.25 | 1.47 | 0.2818 |
| Neutrophil (%) | 1.07 | 1 | 1.15 | 0.0445 |

4C. Enrolled subjects blood collected between 32
and <37 weeks of gestational age.

| | | | | |
|---|---|---|---|---|
| WBC, ×10⁹ | 1.06 | 0.9 | 1.27 | 0.4795 |
| BMI prior to pregnancy | 0.96 | 0.79 | 1.17 | 0.6925 |
| (kg/m²) | | | | |
| Education (>12 year) | 0.42 | 0.16 | 1.07 | 0.0716 |
| Neutrophil (%) | 1.03 | 0.98 | 1.09 | 0.3563 |

4D. All enrolled subjects.

| | | | | |
|---|---|---|---|---|
| WBC, ×10⁹ | 1.16 | 1.05 | 1.29 | 0.0058 |
| BMI prior to pregnancy | 0.99 | 0.88 | 1.11 | 0.8179 |
| (kg/m²) | | | | |
| Education (>12 year) | 0.42 | 0.23 | 0.76 | 0.0048 |
| Neutrophil (%) | 1.06 | 1.01 | 1.11 | 0.0148 |

TABLE 5
Univariate odds ratio analysis of the patient characteristics,
with adjustment of gestational age at blood sample collection.

| | | | | |
|---|---|---|---|---|
| Characteristics | OR | 95CI (L) | 95CI (H) | P value |

5A. Enrolled subjects blood collected <28
weeks of gestational age.

| | | | | |
|---|---|---|---|---|
| WBC, ×10⁹ | 1.19 | 0.93 | 1.63 | 0.1891 |
| BMI prior to pregnancy | 0.71 | 0.34 | 1.2 | 0.2635 |
| (kg/m²) | | | | |
| Education (>12 year) | 0.03 | 0 | 0.46 | 0.0276 |
| Neutrophil (%) | 1.28 | 0.97 | 1.98 | 0.1446 |

5B. Enrolled subjects blood collected between 28
and <32 weeks of gestational age.

| | | | | |
|---|---|---|---|---|
| WBC, ×10⁹ | 1.26 | 1.07 | 1.54 | 0.0104 |
| BMI prior to pregnancy | 1.03 | 0.88 | 1.21 | 0.7232 |
| (kg/m²) | | | | |
| Education (>12 year) | 0.61 | 0.25 | 1.45 | 0.2739 |
| Neutrophil (%) | 1.07 | 1.01 | 1.15 | 0.0415 |

5C. Enrolled subjects blood collected between 32
and <37 weeks of gestational age.

| | | | | |
|---|---|---|---|---|
| WBC, ×10⁹ | 1.06 | 0.9 | 1.27 | 0.4747 |
| BMI prior to pregnancy | 0.96 | 0.78 | 1.17 | 0.6665 |
| (kg/m²) | | | | |
| Education (>12 year) | 0.41 | 0.16 | 1.04 | 0.0651 |
| Neutrophil (%) | 1.03 | 0.98 | 1.09 | 0.3596 |

5D. All enrolled subjects.

| | | | | |
|---|---|---|---|---|
| WBC, ×10⁹ | 1.17 | 1.06 | 1.31 | 0.0035 |
| BMI prior to pregnancy | 0.99 | 0.88 | 1.11 | 0.8405 |
| (kg/m²) | | | | |
| Education (>12 year) | 0.45 | 0.24 | 0.83 | 0.0107 |
| Neutrophil (%) | 1.06 | 1.01 | 1.11 | 0.0155 |

TABLE 6
Multivariate odds ratio analysis of the patient characteristics,
without adjustment of gestational age at blood sample collection.

| | | | | |
|---|---|---|---|---|
| Characteristics | OR | 95CI (L) | 95CI (H) | P value |

6A. Enrolled subjects blood collected <28
weeks of gestational age.

| | | | | |
|---|---|---|---|---|
| WBC, ×10⁹ | 1.23 | 0.84 | 2.09 | 0.3288 |
| BMI PRIOR TO | 0.67 | 0.19 | 1.53 | 0.4246 |
| PREGNANCY | | | | |
| (KG/M²) | | | | |
| EDUCATION (>12 | 0.06 | 0 | 1.49 | 0.1256 |
| YEAR) | | | | |
| NEUTROPHIL (%) | 0.91 | 0.54 | 1.44 | 0.6742 |

6B. Enrolled subjects blood collected between 28
and <32 weeks of gestational age.

| | | | | |
|---|---|---|---|---|
| WBC, ×10⁹ | 1.26 | 1.03 | 1.59 | 0.0359 |
| BMI PRIOR TO | 1.01 | 0.83 | 1.22 | 0.954 |
| PREGNANCY | | | | |
| (KG/M²) | | | | |
| EDUCATION (>12 | 0.57 | 0.2 | 1.54 | 0.2716 |
| YEAR) | | | | |
| NEUTROPHIL (%) | 1.04 | 0.96 | 1.14 | 0.3687 |

6C. Enrolled subjects blood collected between 32
and <37 weeks of gestational age.

| | | | | |
|---|---|---|---|---|
| WBC, ×10⁹ | 1.09 | 0.9 | 1.33 | 0.4066 |
| BMI PRIOR TO | 0.94 | 0.75 | 1.17 | 0.5723 |
| PREGNANCY | | | | |
| (KG/M²) | | | | |
| EDUCATION (>12 | 0.55 | 0.2 | 1.52 | 0.2488 |
| YEAR) | | | | |
| NEUTROPHIL (%) | 1.02 | 0.97 | 1.09 | 0.4112 |

6D. All enrolled subjects.

| | | | | |
|---|---|---|---|---|
| WBC, ×10⁹ | 1.16 | 1.03 | 1.32 | 0.0185 |
| BMI PRIOR TO | 0.96 | 0.84 | 1.1 | 0.5508 |
| PREGNANCY | | | | |
| (KG/M²) | | | | |
| EDUCATION (>12 | 0.46 | 0.24 | 0.89 | 0.0228 |
| YEAR) | | | | |
| NEUTROPHIL (%) | 1.04 | 0.99 | 1.09 | 0.1247 |

TABLE 7
Multivariate odds ratio analysis of the patient characteristics,
with adjustment of gestational age at blood sample collection.

| | | | | |
|---|---|---|---|---|
| Characteristics | OR | 95CI (L) | 95CI (H) | P value |

7A. Enrolled subjects blood collected <28
weeks of gestational age.

| | | | | |
|---|---|---|---|---|
| WBC, ×10⁹ | 1.11 | 0.61 | 1.95 | 0.6739 |
| BMI PRIOR TO | 0.5 | 0.08 | 1.33 | 0.2807 |
| PREGNANCY | | | | |
| (KG/M²) | | | | |
| EDUCATION (>12 | 0.03 | 0 | 1 | 0.0898 |
| YEAR) | | | | |
| NEUTROPHIL (%) | 0.97 | 0.52 | 1.75 | 0.9072 |

7B. Enrolled subjects blood collected between 28
and <32 weeks of gestational age.

| | | | | |
|---|---|---|---|---|
| WBC, ×10⁹ | 1.28 | 1.04 | 1.63 | 0.0302 |
| BMI PRIOR TO | 1.01 | 0.83 | 1.22 | 0.9359 |
| PREGNANCY | | | | |
| (KG/M²) | | | | |
| EDUCATION (>12 | 0.56 | 0.19 | 1.53 | 0.2627 |
| YEAR) | | | | |
| NEUTROPHIL (%) | 1.04 | 0.96 | 1.14 | 0.3565 |

7C. Enrolled subjects blood collected between 32
and <37 weeks of gestational age.

| | | | | |
|---|---|---|---|---|
| WBC, ×10⁹ | 1.1 | 0.9 | 1.35 | 0.3448 |
| BMI PRIOR TO | 0.92 | 0.73 | 1.16 | 0.4843 |
| PREGNANCY | | | | |
| (KG/M²) | | | | |
| EDUCATION (>12 | 0.54 | 0.19 | 1.49 | 0.2315 |
| YEAR) | | | | |
| NEUTROPHIL (%) | 1.02 | 0.97 | 1.09 | 0.4466 |

7D. All enrolled subjects.

| | | | | |
|---|---|---|---|---|
| WBC, ×10⁹ | 1.19 | 1.05 | 1.36 | 0.0079 |
| BMI PRIOR TO | 0.96 | 0.83 | 1.1 | 0.5213 |
| PREGNANCY | | | | |
| (KG/M²) | | | | |
| EDUCATION (>12 | 0.51 | 0.26 | 1.01 | 0.0538 |
| YEAR) | | | | |
| NEUTROPHIL (%) | 1.04 | 0.99 | 1.09 | 0.1267 |

TABLE 8
Univariate hazard ratio analysis of the patient characteristics,
without adjustment of gestational age at blood sample collection.

| | | | | |
|---|---|---|---|---|
| Characteristics | HR | 95CI (L) | 95CI (H) | P value |

8A. Enrolled subjects blood collected <28
weeks of gestational age.

| | | | | |
|---|---|---|---|---|
| WBC, ×10⁹ | 1.29 | 1 | 1.66 | 0.0489 |
| BMI PRIOR TO | 0.93 | 0.58 | 1.48 | 0.7508 |
| PREGNANCY | | | | |
| (KG/M²) | | | | |
| EDUCATION (>12 | 0.09 | 0.01 | 1.07 | 0.0567 |
| YEAR) | | | | |
| NEUTROPHIL (%) | 1.39 | 0.94 | 2.05 | 0.0952 |

8B. Enrolled subjects blood collected between 28
and <32 weeks of gestational age.

| | | | | |
|---|---|---|---|---|
| WBC, ×10⁹ | 1.14 | 1.07 | 1.23 | <0.001 |
| BMI PRIOR TO | 1.05 | 0.93 | 1.18 | 0.4461 |
| PREGNANCY | | | | |
| (KG/M²) | | | | |
| EDUCATION (>12 | 0.86 | 0.47 | 1.56 | 0.6179 |
| YEAR) | | | | |
| NEUTROPHIL (%) | 1.06 | 1.02 | 1.11 | 0.0088 |

8C. Enrolled subjects blood collected between 32
and <37 weeks of gestational age.

| | | | | |
|---|---|---|---|---|
| WBC, ×10⁹ | 1.05 | 0.92 | 1.19 | 0.49 |
| BMI PRIOR TO | 0.98 | 0.85 | 1.13 | 0.7672 |
| PREGNANCY | | | | |
| (KG/M²) | | | | |
| EDUCATION (>12 | 0.65 | 0.34 | 1.27 | 0.2111 |
| YEAR) | | | | |
| NEUTROPHIL (%) | 1.02 | 0.96 | 1.07 | 0.5681 |

8D. All enrolled subjects.

| | | | | |
|---|---|---|---|---|
| WBC, ×10⁹ | 1.16 | 1.08 | 1.23 | <0.001 |
| BMI PRIOR TO | 1.01 | 0.93 | 1.1 | 0.8137 |
| PREGNANCY | | | | |
| (KG/M²) | | | | |
| EDUCATION (>12 | 0.66 | 0.43 | 1.01 | 0.0579 |
| YEAR) | | | | |
| NEUTROPHIL (%) | 1.06 | 1.02 | 1.1 | 0.0014 |

TABLE 9
Univariate hazard ratio analysis of the patient characteristics, with
adjustment of gestational age at blood sample collection.

| | | | | |
|---|---|---|---|---|
| Characteristics | HR | 95CI (L) | 95CI (H) | P value |

9A. Enrolled subjects blood collected <28 weeks of gestational age.

| | | | | |
|---|---|---|---|---|
| WBC, ×10⁹ | 1.27 | 0.98 | 1.65 | 0.0678 |
| BMI PRIOR TO | 0.71 | 0.35 | 1.42 | 0.3273 |
| PREGNANCY | | | | |
| (KG/M²) | | | | |
| EDUCATION (>12 | 0.08 | 0.01 | 0.93 | 0.0433 |
| YEAR) | | | | |
| NEUTROPHIL (%) | 1.93 | 0.97 | 3.84 | 0.0595 |

9B. Enrolled subjects blood collected between 28 and <32 weeks of
gestational age.

| | | | | |
|---|---|---|---|---|
| WBC, ×10⁹ | 1.15 | 1.07 | 1.24 | <0.001 |
| BMI PRIOR TO | 1.05 | 0.93 | 1.18 | 0.4525 |
| PREGNANCY | | | | |
| (KG/M²) | | | | |
| EDUCATION (>12 | 0.86 | 0.47 | 1.59 | 0.6396 |
| YEAR) | | | | |
| NEUTROPHIL (%) | 1.07 | 1.02 | 1.12 | 0.009 |

9C. Enrolled subjects blood collected between 32 and <37 weeks of
gestational age.

| | | | | |
|---|---|---|---|---|
| WBC, ×10⁹ | 1.05 | 0.92 | 1.19 | 0.4953 |
| BMI PRIOR TO | 0.98 | 0.85 | 1.13 | 0.789 |
| PREGNANCY | | | | |
| (KG/M²) | | | | |
| EDUCATION (>12 | 0.65 | 0.34 | 1.27 | 0.2108 |
| YEAR) | | | | |
| NEUTROPHIL (%) | 1.02 | 0.96 | 1.07 | 0.5484 |

9D. All enrolled subjects.

| | | | | |
|---|---|---|---|---|
| WBC, ×10⁹ | 1.16 | 1.09 | 1.23 | <0.001 |
| BMI PRIOR TO | 1.01 | 0.93 | 1.1 | 0.8012 |
| PREGNANCY | | | | |
| (KG/M²) | | | | |
| EDUCATION (>12 | 0.69 | 0.44 | 1.06 | 0.0929 |
| YEAR) | | | | |
| NEUTROPHIL (%) | 1.06 | 1.02 | 1.1 | 0.0019 |

TABLE 10
Multivariate hazard ratio analysis of the patient characteristics, without
adjustment of gestational age at blood sample collection.

| | | | | |
|---|---|---|---|---|
| Characteristics | HR | 95CI (L) | 95CI (H) | P value |

10A. Enrolled subjects blood collected <28 weeks of gestational age.

| | | | | |
|---|---|---|---|---|
| WBC, ×10⁹ | 1.23 | 0.85 | 1.77 | 0.2754 |
| BMI PRIOR TO | 0.79 | 0.33 | 1.88 | 0.5895 |
| PREGNANCY | | | | |
| (KG/M²) | | | | |
| EDUCATION (>12 | 0.42 | 0.02 | 10.27 | 0.5924 |
| YEAR) | | | | |
| NEUTROPHIL (%) | 1.15 | 0.7 | 1.89 | 0.573 |

10B. Enrolled subjects blood collected between 28 and <32 weeks of
gestational age.

| | | | | |
|---|---|---|---|---|
| WBC, ×10⁹ | 1.12 | 1.03 | 1.22 | 0.011 |
| BMI PRIOR TO | 1.04 | 0.91 | 1.18 | 0.5569 |
| PREGNANCY | | | | |
| (KG/M²) | | | | |
| EDUCATION (>12 | 0.8 | 0.41 | 1.56 | 0.5121 |
| YEAR) | | | | |
| NEUTROPHIL (%) | 1.04 | 0.98 | 1.1 | 0.1986 |

10C. Enrolled subjects blood collected between 32 and <37 weeks of
gestational age.

| | | | | |
|---|---|---|---|---|
| WBC, ×10⁹ | 1.08 | 0.94 | 1.25 | 0.2786 |
| BMI PRIOR TO | 0.94 | 0.8 | 1.1 | 0.4584 |
| PREGNANCY | | | | |
| (KG/M²) | | | | |
| EDUCATION (>12 | 0.74 | 0.35 | 1.57 | 0.4322 |
| YEAR) | | | | |
| NEUTROPHIL (%) | 1.01 | 0.95 | 1.06 | 0.7921 |

10D. All enrolled subjects.

| | | | | |
|---|---|---|---|---|
| WBC, ×10⁹ | 1.14 | 1.06 | 1.22 | <0.001 |
| BMI PRIOR TO | 0.98 | 0.89 | 1.08 | 0.713 |
| PREGNANCY | | | | |
| (KG/M²) | | | | |
| EDUCATION (>12 | 0.76 | 0.48 | 1.2 | 0.2397 |
| YEAR) | | | | |
| NEUTROPHIL (%) | 1.03 | 0.99 | 1.08 | 0.0992 |

TABLE 11
Multivariate hazard ratio analysis of the patient characteristics, with
adjustment of gestational age at blood sample collection.

| | | | | |
|---|---|---|---|---|
| Characteristics | HR | 95CI (L) | 95CI (H) | P value |

11A. Enrolled subjects blood collected <28 weeks of gestational age.

| | | | | |
|---|---|---|---|---|
| WBC, ×10⁹ | 1.38 | 0.51 | 3.72 | 0.5209 |
| BMI PRIOR TO | 0.1 | 0 | 34.01 | 0.4369 |
| PREGNANCY | | | | |
| (KG/M²) | | | | |
| EDUCATION (>12 | 0.16 | 0 | 28.93 | 0.492 |
| YEAR) | | | | |
| NEUTROPHIL (%) | 1.65 | 0.69 | 3.91 | 0.2594 |

11B. Enrolled subjects blood collected between 28 and <32 weeks of
gestational age.

| | | | | |
|---|---|---|---|---|
| WBC, ×10⁹ | 1.12 | 1.03 | 1.23 | 0.0094 |
| BMI PRIOR TO | 1.04 | 0.91 | 1.18 | 0.5786 |
| PREGNANCY | | | | |
| (KG/M²) | | | | |
| EDUCATION (>12 | 0.82 | 0.42 | 1.61 | 0.5694 |
| YEAR) | | | | |
| NEUTROPHIL (%) | 1.04 | 0.98 | 1.1 | 0.2034 |

11C. Enrolled subjects blood collected between 32 and <37 weeks of
gestational age.

| | | | | |
|---|---|---|---|---|
| WBC, ×10⁹ | 1.08 | 0.94 | 1.25 | 0.2815 |
| BMI PRIOR TO | 0.94 | 0.8 | 1.1 | 0.465 |
| PREGNANCY | | | | |
| (KG/M²) | | | | |
| EDUCATION (>12 | 0.74 | 0.35 | 1.57 | 0.4348 |
| YEAR) | | | | |
| NEUTROPHIL (%) | 1.01 | 0.95 | 1.07 | 0.7858 |

11D. All enrolled subjects.

| | | | | |
|---|---|---|---|---|
| WBC, ×10⁹ | 1.14 | 1.06 | 1.23 | <0.001 |
| BMI PRIOR TO | 0.98 | 0.89 | 1.09 | 0.7448 |
| PREGNANCY | | | | |
| (KG/M²) | | | | |
| EDUCATION (>12 | 0.8 | 0.5 | 1.27 | 0.3373 |
| YEAR) | | | | |
| NEUTROPHIL (%) | 1.03 | 0.99 | 1.07 | 0.1372 |

TABLE 12
GEO microarray datasets used to identify candidate preterm birth differential expressed genes.

| | Preterm | Gestational | | Control | Gestational | | | Authors and | |
|---|---|---|---|---|---|---|---|---|---|
| No. GEO | case (n=) | age of case | | (n=) | age of control | | Dissection | Published Year | PMID |
| GSE18809 | 5 | <34 | wks | 5 | 38-39 | wks | placenta | Chim S S, Lee W S, | 22496790 |
| | | | | | | | | Ting Y H, et al., 2012 | |
| GSE22490 | 4 | <15 | wks | 6 | <15 | wks | placenta | Rull K, Tomberg K, | 23290504 |
| | | | | | | | | Kõks S, et al., 2013 | |
| GSE14722/ | 11 | 24-36 | wks | 27 | 14-24 | wks | basal plate | Winn V D, Gormley M, | 18818296/ |
| GSE5999 | | | | | | | | Paquet A C, et al. | 17170095 |
| | | | | | | | | 2009./Winn V D, | |
| | | | | | | | | Haimov-Kochman R, | |
| | | | | | | | | Gormley M, Fisher S, | |
| | | | | | | | | 2007 | |
| Total | 20 | | | 38 | | | | | |

TABLE 13
ELISA reagents used to validate the biomarker candidates.

| Gene symbol | Gene name | References PMID | Vendor info | CAT# |
|---|---|---|---|---|
| FLT1 | VEGFR-1 | 24440566/24423299/ | R&D systems | DVR100B |
| | ADAM | 24535301 | | |
| ADAM12 | metallopeptidase | 22927907/22540429/ | R&D systems | DAD120 |
| | domain 12 | 25591790 | | |
| INHBA | inhibin, beta A | 24799891/24134550/ | R&D systems | DAC00B |
| | | 21509823 | | |

TABLE 14
Levels of biomarker analyte (ng/ml) detected at different stage of gestation in
pregnant individuals with preterm birth outcome. Median IRQ values are provided.

| | | Normal | | PTB | |
|---|---|---|---|---|---|
| Analyte | PTB trend | Median (IQR) | Mean (SD) | Median (IQR) | Mean (SD) |

14A. Enrolled subjects blood collected <28 weeks of gestational age.

| | | | | | | | | | |
|---|---|---|---|---|---|---|---|---|---|
| Activin A | Up | 1.36 | (1.11, 1.7) | 1.46 | (0.6) | 2.78 | (2.43, 3.45) | 3.11 | (1.45) |
| ADAM12 | Up | 11.12 | (8.74, 13.85) | 11.8 | (5.18) | 25.86 | (16.25, 33.42) | 23.81 | (12.43) |
| sFlt-1 | Up | 0.04 | (0.03, 0.48) | 0.36 | (0.52) | 0.1 | (0.03, 0.66) | 0.6 | (1.05) |

14B. Enrolled subjects blood collected between 28 and <32 weeks of gestational age.

| | | | | | | | | | |
|---|---|---|---|---|---|---|---|---|---|
| Activin A | Up | 1.56 | (1.03, 2.64) | 1.84 | (1.21) | 3.42 | (2.25, 4.4) | 3.43 | (1.31) |
| ADAM12 | Up | 16.73 | (12.18, 25.11) | 17.88 | (9.9) | 19.37 | (15.56, 28.9) | 22.25 | (10.58) |
| sFlt-1 | Up | 0.56 | (0.05, 2.15) | 1.15 | (1.32) | 1.67 | (0.52, 2.79) | 2.12 | (2.77) |

14C. Enrolled subjects blood collected between 32 and <37 weeks of gestational age.

| | | | | | | | | | |
|---|---|---|---|---|---|---|---|---|---|
| Activin A | Up | 3.04 | (2.44, 4.36) | 3.38 | (1.5) | 5.02 | (4.33, 6.02) | 5.15 | (1.25) |
| ADAM12 | Up | 31.11 | (22.18, 38.71) | 30.43 | (13.46) | 40.03 | (30.3, 50.36) | 42.32 | (14.76) |
| sFlt-1 | Up | 1.12 | (0.67, 1.94) | 1.51 | (1.25) | 2.41 | (1.16, 3.95) | 3.35 | (3.34) |

TABLE 15
Levels of biomarker analyte (ng/ml) detected at different stages of
gestation in pregnant individuals with preterm birth outcome with a
targeted specificity.

| | | | |
|---|---|---|---|
| Specificity level | Activin A | ADAM12 | sFlt-1 |

15A. Enrolled subjects blood collected <28 weeks of gestational age.

| | | | |
|---|---|---|---|
| 1 | 0.25 | 0.5 | 0.25 |
| 0.75 | 1 | 0.75 | 0.25 |
| 0.5 | 1 | 0.75 | 0.5 |

15B. Enrolled subjects blood collected between 28 and <32 weeks of
gestational age.

| | | | |
|---|---|---|---|
| 1 | 0.0179 | 0.0893 | 0.0536 |
| 0.95 | 0.4286 | 0.0893 | 0.1429 |
| 0.9 | 0.5536 | 0.1786 | 0.25 |
| 0.85 | 0.6071 | 0.1964 | 0.3036 |
| 0.8 | 0.6429 | 0.2679 | 0.3214 |

15C. Enrolled subjects blood collected between 32 and <37 weeks of
gestational age.

| | | | |
|---|---|---|---|
| 1 | 0.2143 | 0.1905 | 0.0952 |
| 0.95 | 0.2857 | 0.1905 | 0.3333 |
| 0.9 | 0.3095 | 0.2619 | 0.4048 |
| 0.85 | 0.4286 | 0.3095 | 0.4762 |
| 0.8 | 0.5952 | 0.4524 | 0.5238 |

15D. Enrolled subjects blood collected between 28 and <37 weeks of
gestational age.

| Sensitivity level | Activin A | ADAM12 | sFlt-1 |
|---|---|---|---|
| 1 | 0.0882 | 0.0784 | 0.0588 |
| 0.95 | 0.1373 | 0.1176 | 0.2745 |
| 0.9 | 0.3627 | 0.2549 | 0.3039 |
| 0.85 | 0.5784 | 0.3627 | 0.3627 |
| 0.8 | 0.6275 | 0.3922 | 0.4608 |

TABLE 16
Univariate odds ratio analysis of the levels of biomarker analytes, without
adjustment of gestational age at blood sample collection.

| | | | | |
|---|---|---|---|---|
| Marker | OR | 95CI.L | 95CI.H | P value |

16A. Enrolled subjects blood collected <28 weeks of gestational age.

| | | | | |
|---|---|---|---|---|
| Activin A | 9.12 | 1.72 | 157.6 | 0.047 |
| ADAM12 | 1.2 | 1.04 | 1.5 | 0.0403 |
| sFlt-1 | 1.75 | 0.28 | 9.27 | 0.4992 |

16B. Enrolled subjects blood collected between 28 and <32 weeks of
gestational age.

| | | | | |
|---|---|---|---|---|
| Activin A | 2.82 | 1.86 | 4.66 | <0.001 |
| ADAM12 | 1.04 | 1 | 1.09 | 0.051 |
| sFlt-1 | 1.38 | 1.05 | 1.92 | 0.0408 |

16C. Enrolled subjects blood collected between 32 and <37 weeks of
gestational age.

| | | | | |
|---|---|---|---|---|
| Activin A | 2.58 | 1.72 | 4.29 | <0.001 |
| ADAM12 | 1.06 | 1.03 | 1.11 | 0.0017 |
| sFlt-1 | 1.58 | 1.18 | 2.33 | 0.0094 |

16D. Enrolled subjects blood collected between 28 and <37 weeks of
gestational age.

| | | | | |
|---|---|---|---|---|
| Activin A | 2.08 | 1.67 | 2.66 | <0.001 |
| ADAM12 | 1.04 | 1.02 | 1.07 | <0.001 |
| sFlt-1 | 1.52 | 1.24 | 1.92 | <0.001 |

TABLE 17
Univariate odds ratio analysis of the levels of biomarker analytes, with
adjustment of gestational age at blood sample collection.

| | | | | |
|---|---|---|---|---|
| Marker | OR | 95CI.L | 95CI.H | P value |

17A. Enrolled subjects blood collected <28 weeks of gestational age.

| | | | | |
|---|---|---|---|---|
| Activin A | 30.06 | 2.06 | 8702.66 | 0.0856 |
| ADAM12 | 1.24 | 1.04 | 1.71 | 0.0605 |
| sFlt-1 | 2.18 | 0.34 | 16.02 | 0.3751 |

17B. Enrolled subjects blood collected between 28 and <32 weeks of
gestational age.

| | | | | |
|---|---|---|---|---|
| Activin A | 2.83 | 1.86 | 4.69 | <0.002 |
| ADAM12 | 1.04 | 1 | 1.09 | 0.0579 |
| sFlt-1 | 1.38 | 1.04 | 1.95 | 0.0548 |

17C. Enrolled subjects blood collected between 32 and <37 weeks of
gestational age.

| | | | | |
|---|---|---|---|---|
| Activin A | 2.58 | 1.71 | 4.29 | <0.001 |
| ADAM12 | 1.06 | 1.03 | 1.11 | 0.0017 |
| sFlt-1 | 1.69 | 1.21 | 2.61 | 0.0082 |

17D. Enrolled subjects blood collected between 28 and <37 weeks of
gestational age.

| | | | | |
|---|---|---|---|---|
| Activin A | 2.36 | 1.81 | 3.2 | <0.001 |
| ADAM12 | 1.04 | 1.02 | 1.07 | 0.0011 |
| sFlt-1 | 1.48 | 1.2 | 1.9 | <0.001 |

TABLE 18
Multivariate odds ratio analysis of the levels of biomarker analytes,
without adjustment of gestational age at blood sample collection.

| | | | | |
|---|---|---|---|---|
| Marker | OR | 95CI.L | 95CI.H | P value |

18A. Enrolled subjects blood collected <28 weeks of gestational age.

| | | | | |
|---|---|---|---|---|
| Activin A | 9.83 | 0.57 | 757.78 | 0.1657 |
| ADAM12 | 0.99 | 0.72 | 1.38 | 0.9379 |
| sFlt-1 | 0.3 | 0 | 17.02 | 0.5611 |

18B. Enrolled subjects blood collected between 28 and <32 weeks of
gestational age.

| | | | | |
|---|---|---|---|---|
| Activin A | 3.26 | 1.97 | 6.03 | <0.001 |
| ADAM12 | 0.96 | 0.89 | 1.02 | 0.2047 |
| sFlt-1 | 1.22 | 0.85 | 1.83 | 0.3005 |

18C. Enrolled subjects blood collected between 32 and <37 weeks of
gestational age.

| | | | | |
|---|---|---|---|---|
| Activin A | 2.51 | 1.42 | 4.89 | 0.0032 |
| ADAM12 | 1 | 0.94 | 1.05 | 0.9349 |
| sFlt-1 | 1.06 | 0.77 | 1.62 | 0.7673 |

18D. Enrolled subjects blood collected between 28 and <37 weeks of
gestational age.

| | | | | |
|---|---|---|---|---|
| Activin A | 2.57 | 1.82 | 3.78 | <0.001 |
| ADAM12 | 0.96 | 0.92 | 0.99 | 0.029 |
| sFlt-1 | 1.19 | 0.93 | 1.56 | 0.1982 |

TABLE 19
Multivariate odds ratio analysis of the levels of biomarker analytes, with
adjustment of gestational age at blood sample collection.

| | | | | |
|---|---|---|---|---|
| Marker | OR | 95CI.L | 95CI.H | P value |

19A. Enrolled subjects blood collected <28 weeks of gestational age.

| | | | | |
|---|---|---|---|---|
| Activin A | 81.59 | 1.03 | 7390187 | 0.1783 |
| ADAM12 | 0.96 | 0.63 | 1.45 | 0.8402 |
| sFlt-1 | 0.04 | 0 | 5.43 | 0.3236 |

19B. Enrolled subjects blood collected between 28 and <32 weeks of
gestational age.

| | | | | |
|---|---|---|---|---|
| Activin A | 3.27 | 1.97 | 6.03 | <0.001 |
| ADAM12 | 0.96 | 0.89 | 1.02 | 0.2108 |
| sFlt-1 | 1.26 | 0.85 | 1.93 | 0.267 |

19C. Enrolled subjects blood collected between 32 and <37 weeks of
gestational age.

| | | | | |
|---|---|---|---|---|
| Activin A | 2.5 | 1.41 | 4.89 | 0.0034 |
| ADAM12 | 1 | 0.94 | 1.05 | 0.9327 |
| sFlt-1 | 1.06 | 0.76 | 1.72 | 0.772 |

19D. Enrolled subjects blood collected between 28 and <37 weeks of
gestational age.

| | | | | |
|---|---|---|---|---|
| Activin A | 2.71 | 1.89 | 4.04 | <0.001 |
| ADAM12 | 0.96 | 0.93 | 1 | 0.0727 |
| sFlt-1 | 1.23 | 0.95 | 1.63 | 0.1393 |

TABLE 20
Univariate hazard ratio analysis of the levels of biomarker analytes,
without adjustment of gestational age at blood sample collection.

| | | | | |
|---|---|---|---|---|
| Marker | HR | 95CI.L | 95CI.H | P value |

20A. Enrolled subjects blood collected <28 weeks of gestational age.

| | | | | |
|---|---|---|---|---|
| Activin A | 3.11 | 1.24 | 7.79 | 0.0152 |
| ADAM12 | 1.18 | 1.03 | 1.36 | 0.0196 |
| sFlt-1 | 2.28 | 0.5 | 10.37 | 0.2873 |

20B. Enrolled subjects blood collected between 28 and <32 weeks of
gestational age.

| | | | | |
|---|---|---|---|---|
| Activin A | 1.55 | 1.3 | 1.85 | <0.001 |
| ADAM12 | 1.03 | 1 | 1.06 | 0.0278 |
| sFlt-1 | 1.2 | 1.07 | 1.35 | 0.0024 |

20C. Enrolled subjects blood collected between 32 and <37 weeks of
gestational age.

| | | | | |
|---|---|---|---|---|
| Activin A | 1.66 | 1.32 | 2.09 | <0.001 |
| ADAM12 | 1.03 | 1.01 | 1.05 | 0.0026 |
| sFlt-1 | 1.14 | 1.04 | 1.24 | 0.0032 |

20D. Enrolled subjects blood collected between 28 and <37 weeks of
gestational age.

| | | | | |
|---|---|---|---|---|
| Activin A | 1.44 | 1.28 | 1.62 | <0.001 |
| ADAM12 | 1.02 | 1.01 | 1.04 | <0.001 |
| sFlt-1 | 1.14 | 1.07 | 1.22 | <0.001 |

TABLE 21
Univariate hazard ratio analysis of the levels of biomarker analytes, with
adjustment of gestational age at blood sample collection.

| | | | | |
|---|---|---|---|---|
| Marker | HR | 95CI.L | 95CI.H | P value |

21A. Enrolled subjects blood collected <28 weeks of gestational age.

| | | | | |
|---|---|---|---|---|
| Activin A | 2.9 | 1.11 | 7.58 | 0.0296 |
| ADAM12 | 1.18 | 1.01 | 1.37 | 0.0351 |
| sFlt-1 | 3.11 | 0.78 | 12.41 | 0.1075 |

21B. Enrolled subjects blood collected between 28 and <32 weeks of
gestational age.

| | | | | |
|---|---|---|---|---|
| Activin A | 1.7 | 1.39 | 2.09 | <0.001 |
| ADAM12 | 1.03 | 1 | 1.06 | 0.0258 |
| sFlt-1 | 1.21 | 1.08 | 1.36 | 0.0014 |

21C. Enrolled subjects blood collected between 32 and <37 weeks of
gestational age.

| | | | | |
|---|---|---|---|---|
| Activin A | 1.66 | 1.32 | 2.09 | <0.001 |
| ADAM12 | 1.03 | 1.01 | 1.06 | 0.0024 |
| sFlt-1 | 1.15 | 1.06 | 1.26 | 0.0015 |

21D. Enrolled subjects blood collected between 28 and <37 weeks of
gestational age.

| | | | | |
|---|---|---|---|---|
| Activin A | 1.58 | 1.37 | 1.83 | <0.001 |
| ADAM12 | 1.02 | 1.01 | 1.04 | 0.0024 |
| sFlt-1 | 1.14 | 1.07 | 1.22 | <0.001 |

TABLE 22
Multivariate hazard ratio analysis of the levels of biomarker analytes,
without adjustment of gestational age at blood sample collection.

| | | | | |
|---|---|---|---|---|
| Marker | HR | 95CI.L | 95CI.H | P value |

22A. Enrolled subjects blood collected <28 weeks of gestational age.

| | | | | |
|---|---|---|---|---|
| Activin A | 6.65 | 0.19 | 236.47 | 0.2983 |
| ADAM12 | 1.05 | 0.82 | 1.35 | 0.7005 |
| sFlt-1 | 0.18 | 0 | 7.28 | 0.367 |

22B. Enrolled subjects blood collected between 28 and <32 weeks of
gestational age.

| | | | | |
|---|---|---|---|---|
| Activin A | 1.62 | 1.25 | 2.1 | <0.001 |
| ADAM12 | 0.99 | 0.95 | 1.02 | 0.4005 |
| sFlt-1 | 1.02 | 0.87 | 1.2 | 0.8003 |

22C. Enrolled subjects blood collected between 32 and <37 weeks of
gestational age.

| | | | | |
|---|---|---|---|---|
| Activin A | 2.01 | 1.32 | 3.04 | 0.001 |
| ADAM12 | 0.98 | 0.95 | 1.02 | 0.3285 |
| sFlt-1 | 0.98 | 0.87 | 1.1 | 0.719 |

22D. Enrolled subjects blood collected between 28 and <37 weeks of
gestational age.

| | | | | |
|---|---|---|---|---|
| Activin A | 1.8 | 1.45 | 2.22 | <0.001 |
| ADAM12 | 0.97 | 0.95 | 0.99 | 0.0073 |
| sFlt-1 | 1 | 0.91 | 1.08 | 0.9106 |

TABLE 23
Multivariate hazard ratio analysis of the levels of biomarker analytes, with
adjustment of gestational age at blood sample collection.

| | | | | |
|---|---|---|---|---|
| Marker | HR | 95CI.L | 95CI.H | P value |

23A. Enrolled subjects blood collected <28 weeks of gestational age.

| | | | | |
|---|---|---|---|---|
| Activin A | 30.26 | 0.04 | 20453.46 | 0.3051 |
| ADAM12 | 1.02 | 0.78 | 1.33 | 0.885 |
| sFlt-1 | 0.03 | 0 | 38.07 | 0.3469 |

23B. Enrolled subjects blood collected between 28 and <32 weeks of
gestational age.

| | | | | |
|---|---|---|---|---|
| Activin A | 1.76 | 1.33 | 2.31 | <0.001 |
| ADAM12 | 0.99 | 0.95 | 1.02 | 0.4557 |
| sFlt-1 | 1.03 | 0.9 | 1.18 | 0.6667 |

23C. Enrolled subjects blood collected between 32 and <37 weeks of
gestational age.

| | | | | |
|---|---|---|---|---|
| Activin A | 1.97 | 1.3 | 3 | 0.0014 |
| ADAM12 | 0.98 | 0.95 | 1.02 | 0.3198 |
| sFlt-1 | 0.99 | 0.87 | 1.12 | 0.8629 |

23D. Enrolled subjects blood collected between 28 and <37 weeks of
gestational age.

| | | | | |
|---|---|---|---|---|
| Activin A | 1.88 | 1.51 | 2.34 | <0.001 |
| ADAM12 | 0.97 | 0.95 | 1 | 0.0247 |
| sFlt-1 | 0.99 | 0.91 | 1.08 | 0.84 |

TABLE 24
ROC AUC of each biomarker analyte at different blood sample
collection during the gestational age.

| Panel^{a} | <28 weeks | 28 to <32 weeks | 32 to <37 weeks | Overall |
|---|---|---|---|---|
| Activin A | 0.9062 | 0.8374 | 0.8143 | 0.7993 |
| ADAM12 | 0.7812 | 0.6175 | 0.7082 | 0.6678 |
| sFlt-1 | 0.5156 | 0.6231 | 0.702 | 0.672 |

### Results

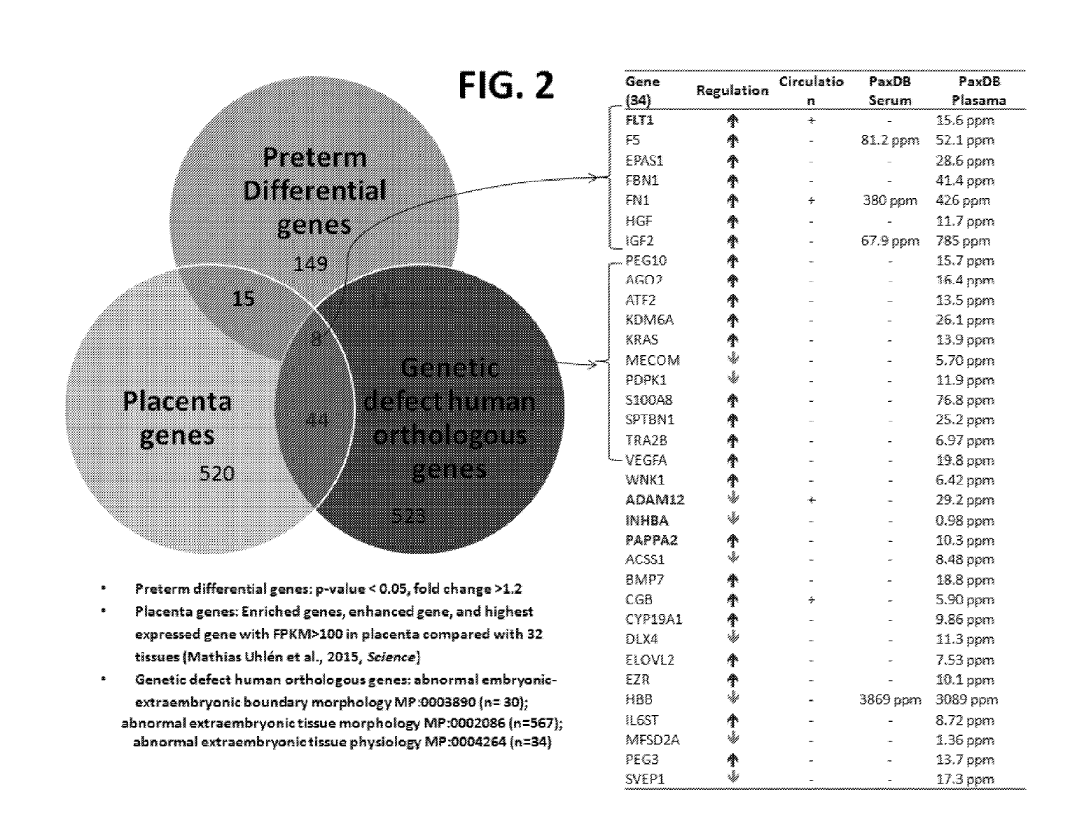

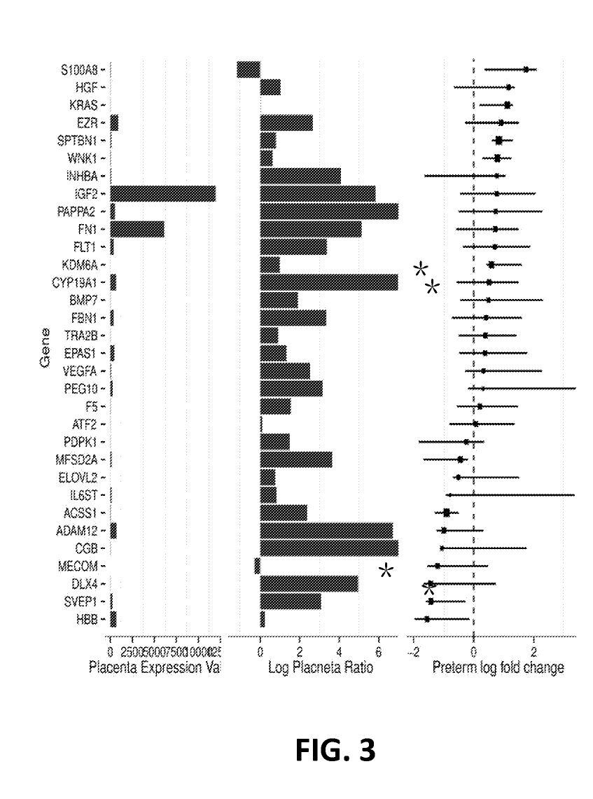

Multi-‘Omics’-based discovery revealing preterm birth marker candidates. As shown in FIGS. 1 and 2 and Table 12, previous placental expression studies were combined for a multiplex meta-analysis, as well as Protein Atlas analysis and human orthologous gene analysis, to discover biomarker candidates diagnosing preterm birth from normal controls. This effort identified Activin A, placenta growth factor, fibronectin 1, paternally expressed 10, Adam12, and sFlt-1 as differential placental biomarkers for preterm birth.

Sample characteristics. The preterm birth case and control subjects used for serological protein biomarker validation can be divided into early (case, n=4; control, n=16 with gestation weeks early than 28), 28 to <32 weeks (case, n=56; control, n=38), and 32 to <37 weeks (case, n=42; control, n=35). As summarized in Table 1-11, patient demographics were shown.

Analysis on risk factors of preterm birth. A group of risk factors including BMI, whole blood cell count, education level, and neutrophil percentage during pregnancy were selected by literature review. The impact of these risk factors was investigated by univariate and multivariate analysis at early, late, and overall stages, with or without adjustment of gestational age at blood sample collection, respectively (Tables 1-11). Results of univariate analysis showed that education level, whole blood cell count, and neutrophil differential abnormality (p<0.05) are potential risk factors on preterm birth.

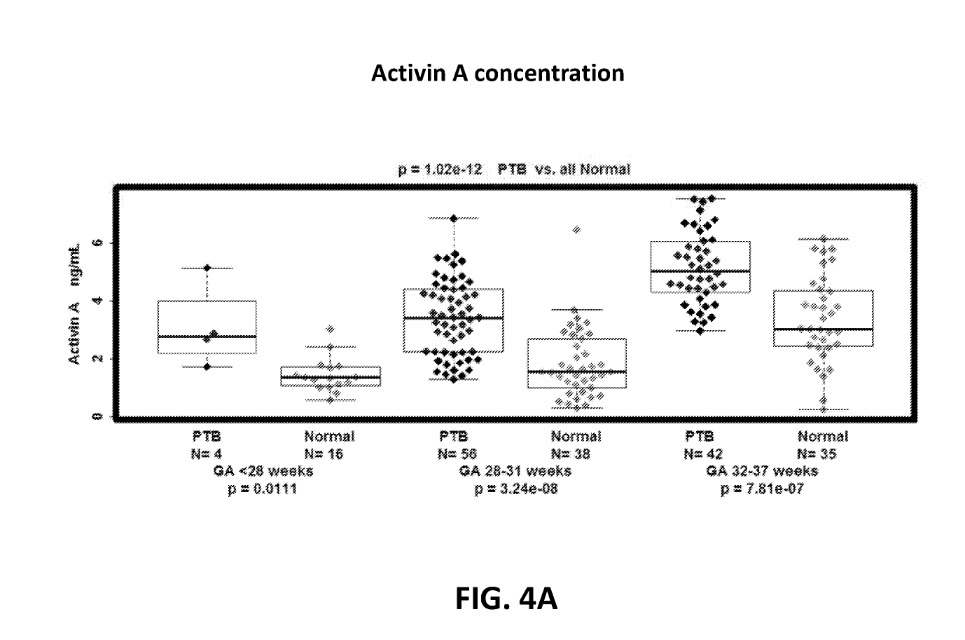

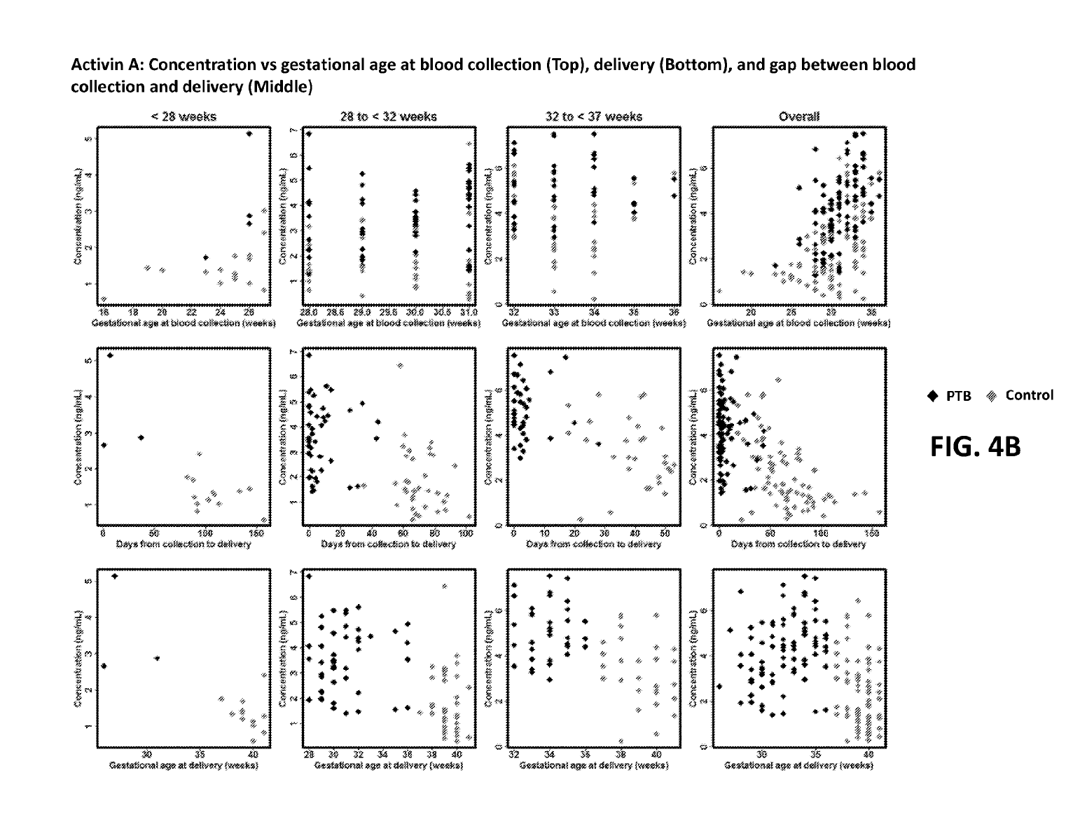

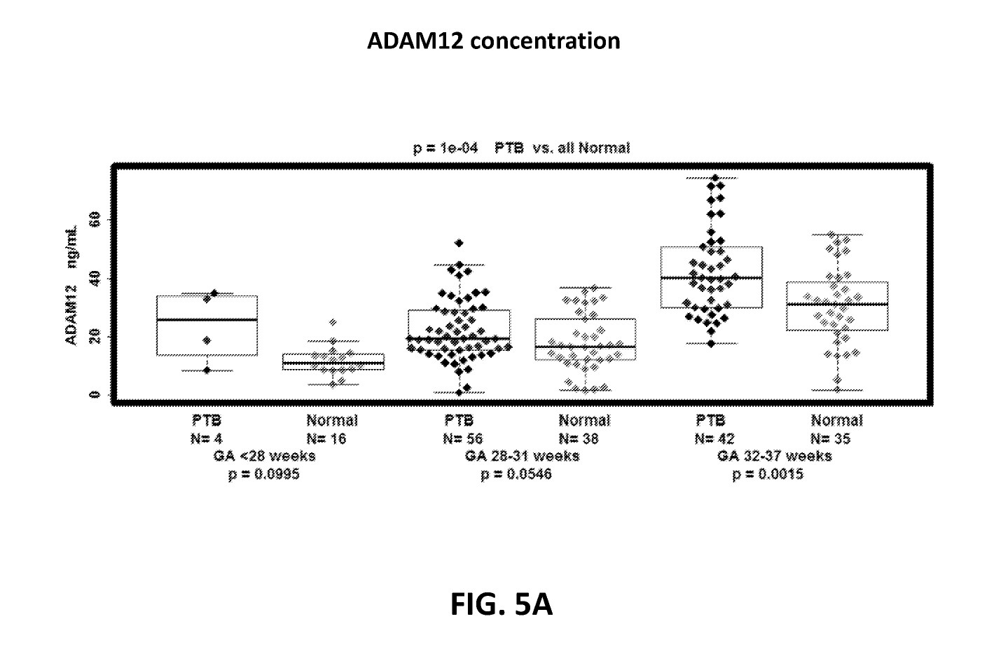

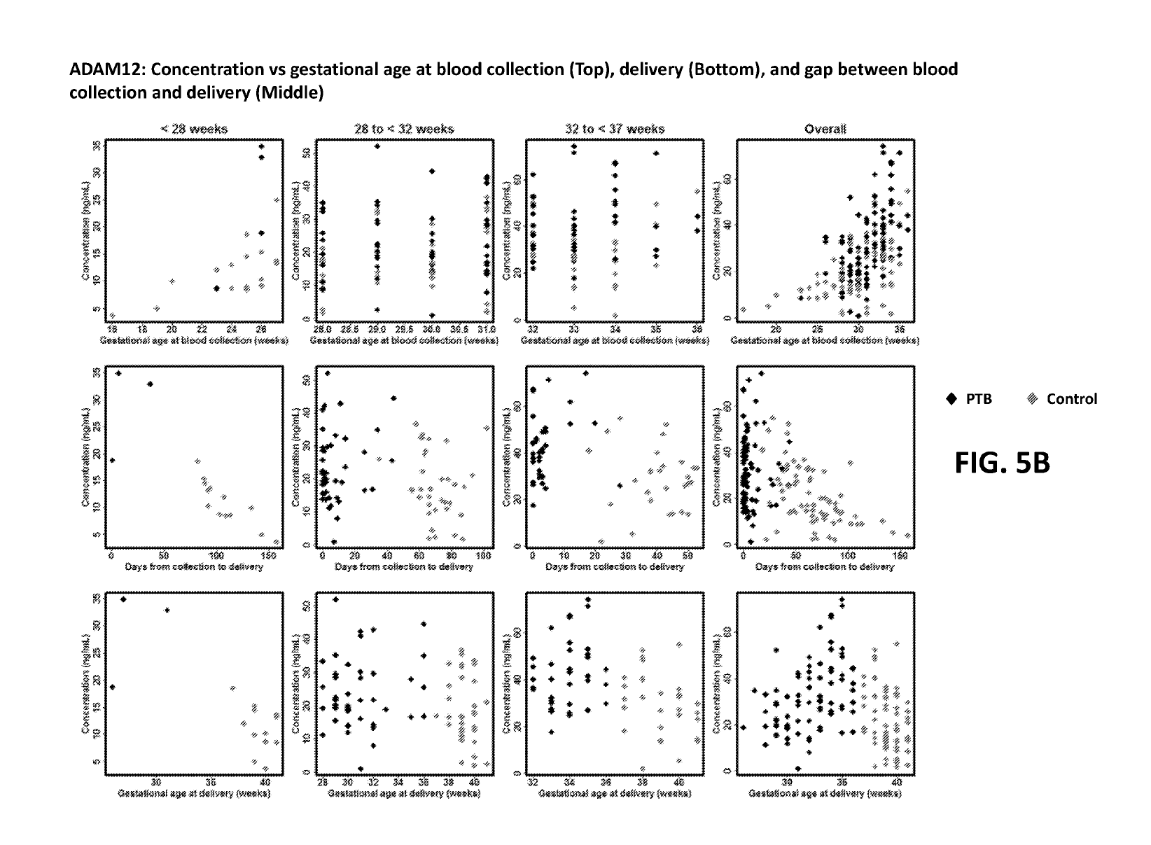

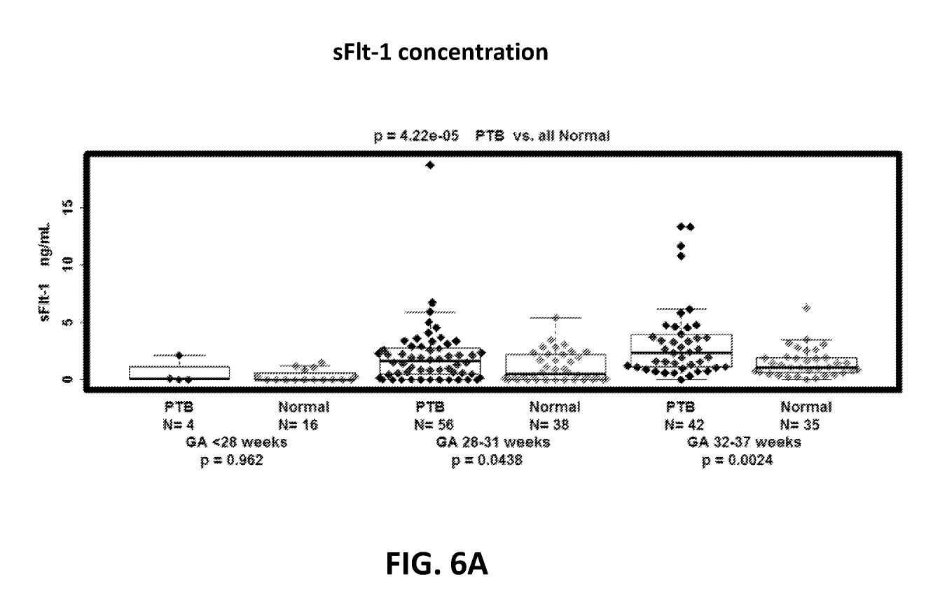

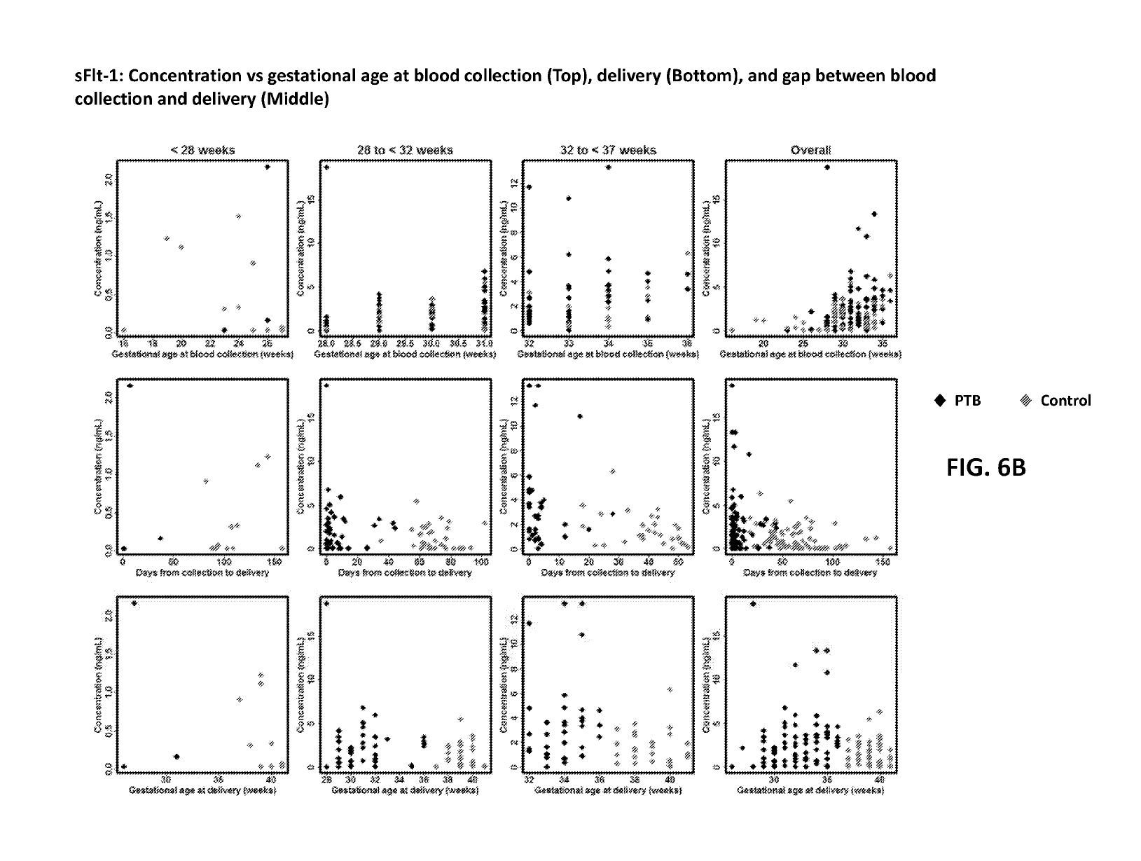

Biomarker validation using preterm birth and control maternal serum samples. To identify whether the preterm serological protein panel could enable development of an immediate practical clinical tool, based on available ELISA assays, biomarker candidates, from expression meta-analysis, proteomics analysis and mouse genetics phenotypic analysis, were validated with available serum assays using preterm birth (n=102) and gestation age-matched control samples (n=89). Detailed with whisker box and scatter plots in FIGS. 4-6, three proteins were validated by ELISA assays (Mann-Whitney U-test). FIGS. 4-6 also demonstrated the distribution of maternal serum abundance of each validated protein over the gestational age (weeks) of blood sample collection, delivery, and the gap in between. Each validated biomarker's median, mean and standard deviation of maternal serum abundance, in preterm birth and control samples, are summarized in Table 14 and FIGS. 4-6.

Univariate and multivariate analysis of validated preterm birth biomarkers. Tables 15-24 summarize the preterm to control hazard ratio, odds ratio and predictive performance early and late gestation maternal serum analyses.

Preterm birth biomarker panel construction. Using data from the ELISA assays, we constructed a random forest algorithm of the three protein analyte panel (Activin A, Adam12 and sFlt1). We sought to identify biomarker panels of optimal feature number, balancing the need for small panel size, accuracy of classification, goodness of class separation (case versus control), and sufficient sensitivity and specificity.

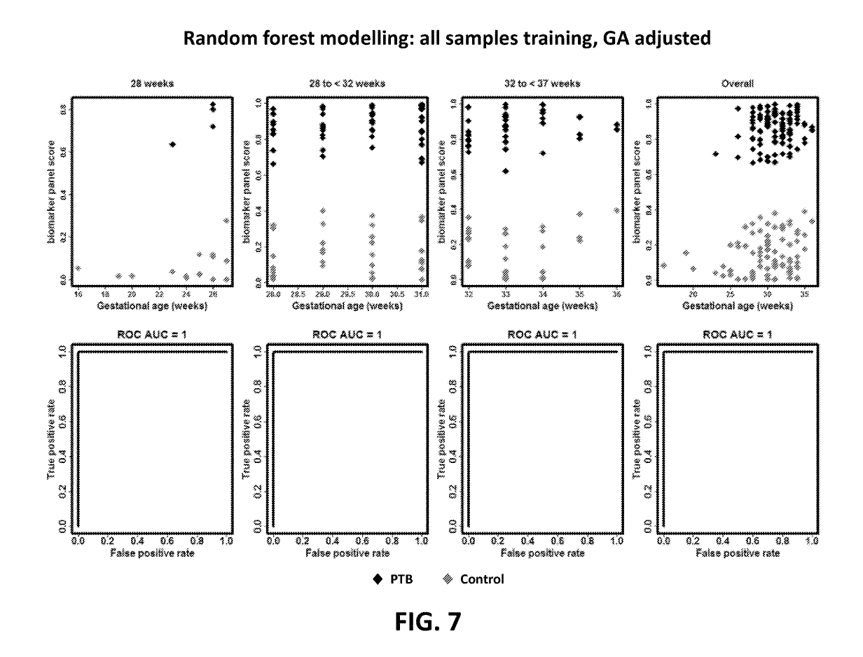

To demonstrate the efficacy of the biomarker panel as a classifier for preterm birth disease activity according to disease onset, the biomarker panel scores were plotted as a function of time of the gestational age (details shown in FIG. 7).

Pathway analysis of preterm birth biomarkers. We analyzed the validated biomarkers that are significantly differentially expressed in preterm birth as a composite, using PathVisio software (version 3.2.1, an open-source pathway analysis and drawing software) (Martijn et al. Presenting and exploring biological pathways with PathVisio. BMC Bioinformatics 2008; 9 (1): 399). In addition to the angiogenesis and focal adhesion pathway involved well-studied angiogenesis biomarker FLT1, our pathway analysis led to the identification of the following statistically significant canonical pathways which may play important roles in preterm birth pathophysiology: activin A is a homodimeric protein that consists of 2 βA subunits. Activin A is a member of the transforming growth factor-β family and is related to inhibin A. It has pleotropic actions, including the stimulation of follicle-stimulating hormone release in the anterior pituitary, a role in neuronal health, and an effect on body axis development; it is produced in a variety of tissues, such as brain, pituitary gland, gonads, bone marrow, and placenta; evidence that supports an activin A role in pregnancy results from both in vitro and clinical studies. All of these support our hypothesis that preterm birth is associated with increased shedding of placental debris leading to increased plasma levels of biomarker proteins, which could contribute to the inflammatory response, hormone imbalance, and endothelial dysfunction.

We have applied a multi-‘omics’ approach to develop validated preterm biomarkers, integrating discoveries from placental mRNA expression meta-analysis, proteomics and mouse genetics phenotypic analyses. Comparing preterm birth and control sera with commercially available ELISA assays, we have validated 3 protein markers, including Activin A, sFlt-1 and Adam12 in predicting preterm birth. The concept of combining a transcriptomic approach in placenta tissue with a proteomic approach, and integration of mouse genetics phenotypes to identify protein biomarkers in serum is novel. It combines the advantages of a study in tissue which is closer to the focus of the pathophysiology with those of a study in serum which is more appropriate for clinical use. Taking proteins that have been discovered/predicted from the discovery phase to an ELISA-based validation phase makes the findings of this study translatable into clinical practice.

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for identifying a pregnant woman as at risk of preterm birth, comprising:
(a) measuring, in a biological sample isolated from the woman, an expression representation of gene Activin A (inhibin beta A or INHBA); and
(b) identifying the woman as at risk of preterm birth if the expression representation of Activin A is upregulated, wherein the upregulation is as compared to a control pregnant woman not at risk of preterm birth.

2. The method of claim 1, wherein the method further comprises
(a) measuring, in the biological sample isolated from the woman, an expression representation of at least one gene selected from Adam12 (ADAM metallopeptidase domain 12) and sFlt1 (fms related tyrosine kinase 1); and
(b) identifying the woman as at risk of preterm birth if the expression representation of Activin A and of any one or both of Adam12 and sFlt1 is upregulated, wherein the upregulation is as compared to a control pregnant woman not at risk of preterm birth.

3. The method of claim 1, wherein the expression representation is a representation of an expression level of a gene at the RNA or protein level.

4. The method of claim 1, wherein the measurement is performed for no more than six genes for the woman.

5. The method of claim 1, wherein the measurement is performed for no more than four genes for the woman.

6. The method of claim 1, wherein the measurement is only performed for Activin A, Adam12 and sFlt1.

7. The method of claim 1, wherein the method does not include measuring the expression levels of any of FN1, PEG10, or PAPPA2.

8. The method of claim 1, wherein the method does not include measuring the expression levels of any of FN1, PEG10, PAPPA2, EPAS1, F5, FBN1, HGF, IGF2, AGO2, ATF2, KDM6A, KRAS, MECOM, PDPK1, S100A8, SPTBN1, TRA2B, VEGFA, WNK1, ACSS1, BMP7, CGB, CYP19A1, DLX4, ELOVL2, EZR, HBB, IL6ST, MFSD2A, PEG3, or SVEP1.

9. The method of claim 1, wherein the woman is pregnant for 16-27 weeks, 28-31 weeks, or 32-36 weeks.

10-11. (canceled)

12. The method of claim 1, wherein the measurement is performed with antibodies directed at proteins expressed by the genes.

13. The method of claim 1, wherein the measurement is performed with oligonucleotides directed at nucleic acid transcripts of the genes.

14. The method of claim 1, wherein the woman:
(a) smokes or consumes alcohol;
(b) is younger than 17 or older than 35;
(c) has preterm birth history; or
(d) is stressed or unhealthy.

15. The method of claim 1, wherein the biological sample is serum.

16. A method of treating a pregnant woman as at risk of preterm birth, comprising:
(a) measuring, in a serum sample obtained from the woman, an expression representation of no more than six genes comprising Activin A (inhibin beta A or INHBA), wherein the woman is pregnant for less than 37 weeks; and
(b) administering to the woman a procedure to ameliorate the preterm birth risk when the woman is identified as at risk of preterm birth for having unregulated expression representation of Activin A, wherein the upregulation is as compared to a control pregnant woman not at risk of preterm birth.

17. The method of claim 16, wherein the method further comprises
(a) measuring, in the serum sample obtained from the woman, an expression representation of Adam12 (ADAM metallopeptidase domain 12) and/or sFlt1 (fms related tyrosine kinase 1), wherein the woman is pregnant for less than 37 weeks; and
(b) administering to the woman a procedure to ameliorate the preterm birth risk when the woman is identified as at risk of preterm birth for having unregulated expression representation of Activin A and of any one or both of Adam12 and sFlt1, wherein the upregulation is as compared to a control pregnant woman not at risk of preterm birth.

18. The method of claim 16, wherein the procedure is selected from the group consisting of administration of corticosteroid, magnesium sulfate, an antibiotic, or progestin, and cervical cerclage and combinations thereof.

19. The method of claim 16, wherein the woman is pregnant for less than 35 weeks.

20. The method of claim 16, wherein the measurement is performed only for Activin A, Adam12 and sFlt1.

21. (canceled)

22. The method of claim 16, wherein the measurement is not performed on genes selected from FN1, PEG10, and PAPPA2.

23. A kit or package comprising:
(a) antibodies directed to proteins comprising Activin A (inhibin beta A or INHBA);
(b) reagents for detecting protein expression with the antibodies;
(c) control antibody and/or control sample.

24-31. (canceled)
